(19) United States
(12) Patent Application Publication
(10) Pub. No.: US 2025/0312179 A1
(43) Pub. Date: Oct. 9, 2025

(54) BALLOON EXPANDABLE BIFURCATED STENT GRAFT AND METHODS OF USING SAME

(71) Applicant: Endologix LLC, Irvine, CA (US)

(72) Inventors: Craig Welk, Irvine, CA (US); Jason Daniel Maggard, Irvine, CA (US); Ahmed Albayoumy, Irvine, CA (US)

(73) Assignee: Endologix LLC, Irvine, CA (US)

(21) Appl. No.: 18/873,313

(22) PCT Filed: Jun. 9, 2023
(86) PCT No.: PCT/US2023/025003

§ 371 Date: Dec. 9, 2024

Related U.S. Application Data
(60) Provisional application No. 63/351,286, filed on Jun. 10, 2022.
(60) Provisional application No. 63/409,632, filed on Sep. 23, 2022.

(51) Int. Cl. A61F 2/954; A61F 2/07; A61F 2/958

(52) U.S. Cl. CPC A61F 2/954; A61F 2/07; A61F 2/958

(57) ABSTRACT

In an exemplary embodiment a device for treating a patient, comprises a delivery catheter, a compressed expandable bifurcated stent having a main body portion, a first limb portion, and a second limb portion; and an expansion element pre-loaded in a portion of the stent or delivery device. In an exemplary embodiment, device for treating a patient, comprises a delivery catheter, a compressed expandable bifurcated stent having a main body portion, a first limb portion, and a second limb portion; and at least one balloon pre-loaded in a portion of the stent or delivery device.

### INCORPORATION BY REFERENCE TO ANY PRIORITY APPLICATIONS

This application claims priority under 35 U.S.C. § 119(e) to U.S. Provisional Application No. 63/351,286, filed Jun. 10, 2022, and U.S. Provisional application No. 63/409,632, filed, Sep. 23, 2022, the contents of each of which are incorporated by reference in their entireties.

### BACKGROUND OF THE DISCLOSURE

### Field of the Disclosure

This disclosure relates to bifurcated stent grafts and deployment systems therefor, for example, balloon expanded bifurcated stent grafts and deployment systems therefor.

### Description of the Related Art

Aortoiliac occlusive disease (AIOD) is a variant of peripheral artery disease affecting the infrarenal aorta and iliac arteries. Similar to other arterial diseases, aortoiliac occlusive disease obstructs blood flow to distal organs through narrowed lumens or by embolization of plaques. Current treatments for peripheral artery diseases such as AIOD include surgical bypass, angioplasty, kissing stents, and techniques and devices referred to as covered endovascular reconstruction of aortic bifurcation.

### SUMMARY OF SOME EXEMPLIFYING EMBODIMENTS

Embodiments of a method and a device for treating diseased vasculature of a patient's body, including without limitation the infrarenal aorta and iliac arteries and other bifurcated and non-bifurcated arteries or vessels of the body are disclosed herein. Any embodiments of the systems, methods, and devices disclosed herein are configured to be, or can be configured to be, used in the treatment of any bifurcated and non-bifurcated vasculature in the body. In some embodiments, the method and device include expanding a unibody bifurcated balloon expandable stent graft which can be covered with a graft material across an arterial disease affecting the infrarenal aorta and iliac arteries and seating the unibody bifurcated stent device onto the bifurcation of the aorta.

Disclosed herein are embodiments of a deployment system and method for treating bifurcated and non-bifurcated vessels within the body, including without limitation the infrarenal aorta and iliac arteries. Exemplary embodiments include a balloon capable of expanding a main body and an ipsilateral limb of the bifurcated unibody graft wherein the balloon can be stepped balloon that allows for the full expansion of the stent graft in a single balloon dilation step and wherein the distal portion of the stepped balloon is positioned within the main graft portion of the stent graft and has a larger expanded diameter than a more proximal portion of the balloon which is positioned within the smaller diameter ipsilateral branch of the stent graft.

Disclosed herein are embodiments of a deployment system and method for treating bifurcated and non-bifurcated vessels within the body, including without limitation the infrarenal aorta and iliac arteries. In some embodiments, the system can include an expansion element such as a “bead” (which in certain embodiments can comprise an enlarged section, bulb or protrusion) that can be pre-assembled and loaded with the rest of the device or can be inserted subsequently into the device. The bead can track through the lumen of a crimped (also referred to as compressed) like, such as the contralateral limb and dilate the limb enough to allow for a balloon catheter to be subsequently advanced up and through the center of the limb of the stent graft. The bead can be small enough that it can be removed from the partially expanded contralateral sheath.

### BRIEF DESCRIPTION OF THE FIGURES

FIGS. 1A-1F are exemplary embodiments of a delivery system and a stent device at various deployment stages.

FIGS. 2A-2E depict expansion of a stent device limb according to an exemplary embodiment.

FIGS. 3, 4, and 5 depict expansion of a stent device according to an exemplary embodiment.

FIGS. 6 and 7 are exemplary embodiments of systems with one or more expansion elements and a stent device.

FIGS. 8 and 9 are exemplary embodiments of systems showing expansion of a stent device with an expansion element.

FIG. 10 is an example of an occluded bifurcated vessel.

FIGS. 11-28 are exemplary embodiments of a delivery system with a stent device expansion element and a balloon at various deployment stages.

FIGS. 29A-29D depict expansion of a stent device according to an exemplary embodiment.

FIG. 30 is an exemplary embodiment of a delivery system with a first and a second balloon.

FIGS. 31A-31B is another exemplary embodiment of a delivery system with a first and a second balloon.

FIGS. 32A-32B is yet another exemplary embodiment of a delivery system with a first and a second balloon.

FIG. 33 is a delivery system according to an exemplary embodiment.

### DETAILED DESCRIPTION OF SOME EMBODIMENTS

Disclosed herein are embodiments of a system 100 for treating diseased vasculature in the body, including without limitation aortoiliac occlusive lesions and other conditions at the aortic bifurcation, as well as other bifurcated and non-bifurcated arteries and vessels. Therefore, while certain embodiments are described as treating the aortic bifurcation, any embodiments of the systems, methods, and devices disclosed herein are configured to be, or can be configured to be, used in the treatment of any bifurcated and non-bifurcated vasculature in the body. Some embodiments of the system 100 can have a stent device 120 and a delivery catheter 130. In exemplary embodiments the stent device 120 has a unibody design to avoid complications that are typically present with modular or multipart devices.

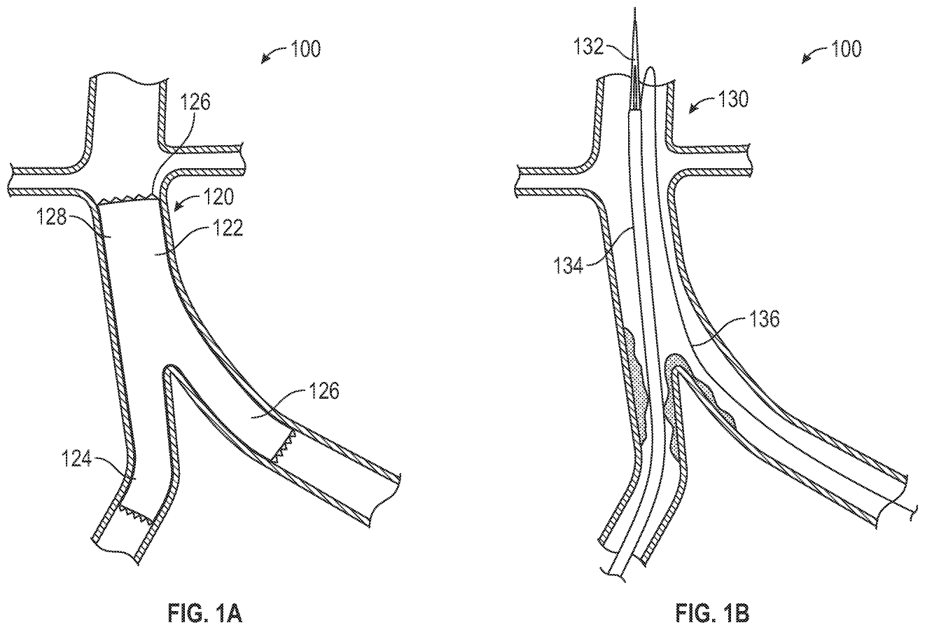

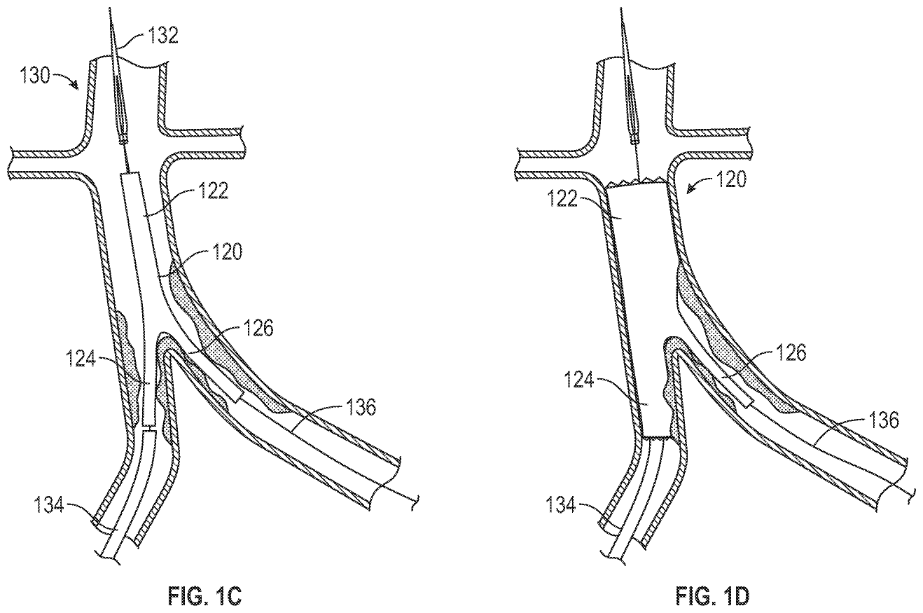

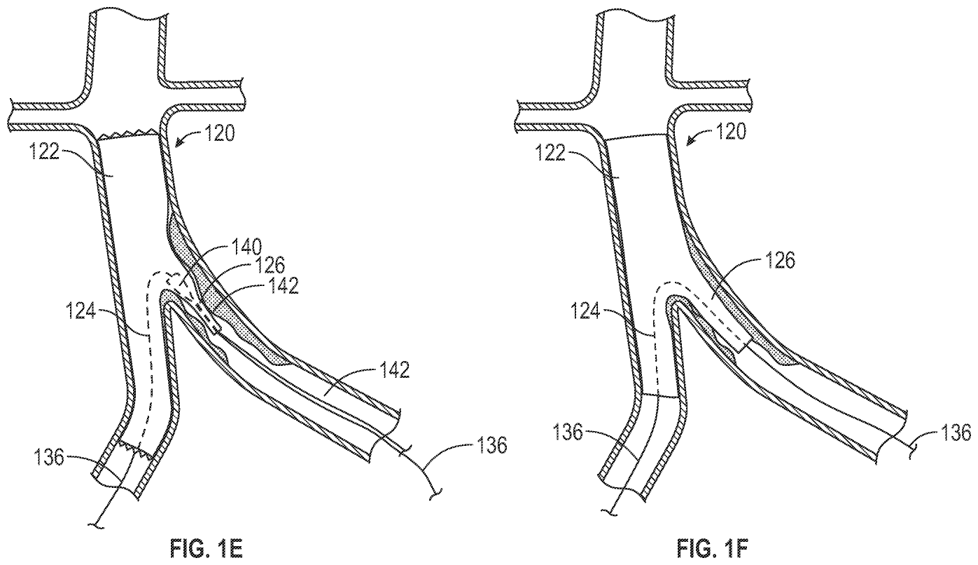

Some embodiments of the methods disclosed herein include deploying a covered bifurcated stent device at the aortic bifurcation, wherein the device has a unibody construction and is mechanically expandable (e.g., balloon expandable). With reference to FIG. 1A, some embodiments of the stent device 120 can have a main body portion 122 that is configured to extend into the aortic artery, a first downwardly (or distally) extending limb portion 124 configured to extend into a first iliac artery (e.g., the ipsilateral or the contralateral artery or limb) and a second downwardly extending limb portion 126 (also referred to herein as the contralimb) configured to extend into a second iliac artery (e.g., the other of the ipsilateral and the contralateral artery or limb). FIG. 1A shows the stent device 120 in a deployed and expanded state. Although some figures show an aneurytic aortic bifurcation, the use of the embodiments disclosed herein are not limited to use for treatment of abdominal aortic aneurysms. The embodiments of the devices disclosed herein can be used to treat a wide range of diseases and conditions of the aorta and aortic bifurcation, including without limitation AIOD and other aneurytic, embolic, and occluded aortic conditions. Some embodiments of the stent device 120 disclosed herein can have the advantage of having a lower profile than some variations of conventional self-expanding stent devices, which can be advantageous when treating certain conditions, such as closed or partially closed aortic arteries.

As will be disclosed in more detail below, any embodiments of the stent device 120 disclosed herein can be an uncovered mechanically expandable (e.g., balloon expandable) stent or a covered mechanically expandable (e.g., balloon expandable) stent of a unibody construction or other one-piece configuration wherein the main body portion 122, the first downwardly extending leg portion 124, and the second downwardly extending leg portion 126 are connected together before deployment. In some embodiments, the main body portion 122, the first downwardly (or distally) extending leg portion 124, and the second downwardly (or distally) extending leg portion 126 can be integrally formed. In some embodiments, the main body portion 122, the first downwardly extending leg portion 124, and the second downwardly extending leg portion 126 can be separately formed and coupled together. In some embodiments, the main body portion 122 and the first downwardly extending leg portion 124 can be integrally formed and the second downwardly extending leg portion 126 can be separately formed and coupled with the main body portion 122 and the first downwardly extending leg portion 124. In some embodiments, the stent device 120 can have an expandable frame 126 and a graft or cover 128.

The frame 126 can have any desired or suitable shape or configuration and can be made from laser cut tubing, wire, or by other known or later developed techniques and materials. In any embodiments, the expandable frame 126 of the stent device 120 can be made from any suitable material, including stainless steel, cobalt chromium, or any other suitable metallic alloy or other material. In any embodiments, the graft can be made from any suitable material for grafts, including polyester, polyester/spandex, expanded polytetrafluoroethylene (ePTFE), or any other suitable or acceptable material. Some embodiments of the stent device 120 can include a balloon expandable bifurcated stent structure that forms an inverted “Y” shape similar to a unibody stent graft structure. In such embodiments, the body portion 122 in the expanded configuration can have a larger external and internal diameter than the downwardly extending leg portions 124, 126. In any embodiments disclosed herein, the expansion balloon can be a stepped balloon to allow for the full expansion of the stent graft in a single balloon dilation step. In such a configuration, a distal portion of the stepped balloon can be positioned within the main graft portion of the stent and have a larger expanded diameter than a more proximal portion of the balloon, which can be positioned within the smaller diameter ipsilateral branch of the stent graft.

With reference to FIG. 1B, some embodiments of the stent device 120 can be deployed using a catheter 130 that can have a distal tip 132 (which can be atraumatic), an outer sheath 134, and a contralateral wire 136. However, in some embodiments an outer catheter sheath 134 is not necessary for the system 100. Some embodiments of the system 100 can be configured such that the stent 120 is crimped in a compressed configuration about an inner core of the delivery catheter 130. Some embodiments of the catheter 130 can be advanced through a puncture site in a first iliac artery through an introducer and can be advanced past the bifurcation of the aorta, or advanced through any puncture site or opening into any portion of a patient's vasculature. The contralateral wire 136 can be withdrawn through a second puncture site in a second of the iliac arteries. With reference to FIG. 1C, the sheath 134 can be withdrawn through the first puncture site, exposing the stent device 120 which can then be moved toward the bifurcation with the first limb portion 124 being withdrawn into the first artery (e.g., without limitation, the ipsilateral iliac artery) and the second limb portion 126 being moved or withdrawn into the second iliac artery (e.g., without limitation, the contralateral iliac artery). The stent device 120 can still be in a contracted or low-profile state at this stage. In some embodiments, the stent 120 can be withdrawn toward the aortic bifurcation so that the bifurcation portion of the stent 120 is moved into contact with the aortic bifurcation, so as to seat the stent 120 at the aortic bifurcation.

With reference to FIG. 1D, the main body portion 122 and the first limb portion 124 can then be expanded, such as with an expansion balloon or other mechanically expandable device. The second limb portion 126 can remain in a collapsed or low-profile state. In some embodiments, the catheter can have an outer sheath. In other embodiments, the catheter can be configured to not have an outer sheath. The outer sheath 134 is shown in FIG. 1D as remaining in the first iliac artery. However, in some embodiments, the outer sheath 134 can be withdrawn before the expansion balloon is inflated or other mechanical expansion means is actuated to expand the main body portion 122 and the first limb portion 124. In some embodiments, an expansion balloon can be positioned within the main body portion 122 and the first limb portion 124 while the stent device 120 is advanced into the aorta. In some embodiments, an expansion balloon can be advanced into the first limb portion 124 and the main body portion 122 after the stent device 120 has been positioned in the desired location in the patient's aorta. As noted above, in certain embodiments, the expansion balloon can be a stepped balloon to allow for the full expansion of the stent graft in a single balloon dilation step. In such a configuration, a distal portion of the stepped balloon can be positioned within the main graft portion of the stent and have a larger expanded diameter than a more proximal portion of the balloon, which can be positioned within the smaller diameter ipsilateral branch of the stent graft. In other embodiments, a single expansion balloon can be used to dilate the main body portion 122 and the first limb portion 124. For example, in a first step, the main body portion 122 can be expanded and then the balloon can be deflated and moved to then dilate the first limb portion 124. In an exemplary embodiment, in a first step, the first limb portion 124 can be expanded first with a balloon and then the balloon can be advanced into the main body portion for expanding the main body portion 122. In addition, while the present disclosure describes the use of a balloon for expanding portions of the device 120, other expansion devices can be used such as mechanically expandable devices.

In some patients, the second iliac artery could be stenotic or otherwise partially or fully closed, making it difficult or impractical to advance an expansion balloon or other mechanical expansion device into the second limb portion 126. To solve this problem, some embodiments of the system 100 can include one or more low profile expansion element(s) 140 that can be moved through the main body portion 122, the first limb portion 124, and/or the second limb portion 126 to partially expand the main body portion 122, the first limb portion 124, and/or the second limb portion 126 so that an expansion balloon or other mechanical expansion means can be advanced into the main body portion 122, the first limb portion 124, and/or the second limb portion 126. For example and without limitation, in some embodiments, the low profile expansion element 140 (also referred to herein as a “bead”) can have a low profile shape that can be moved through the main body portion 122, the first limb portion 124, and/or the second limb portion 126 and cause the main body portion 122, the first limb portion 124, and/or the second limb portion 126 to partially expand despite the forces imparted on the main body portion 122, the first limb portion 124, and/or the second limb portion 126 from the embolic condition of the patient's vasculature. In some embodiments, the low-profile expansion element 140 can have a solid cross-section. In some embodiments, the low-profile expansion element 140 can have an elongated shape, such as a cylindrical shape. In some embodiments, the low-profile expansion element 140 can be pre-assembled and loaded with the rest of the stent device. As discussed, the low-profile expansion element 140 can be moved through the crimped or contracted second limb portion 126 and can dilate the second limb portion 126 enough to allow for a balloon to cannulate up and subsequently fully-expand the second limb portion 126. Some embodiments of the low-profile expansion element 140 can be sized and configured to be withdrawn though a contralateral introducer sheath.

In any embodiments disclosed herein, the low-profile expansion element 140 can be self-expanding or can be actively expanded. A self-expanding bead can be achieved in several manners such as a self-expanding foam, an open cell Polyurethane foam, flexible nitinol membranes, or variations thereof. In an exemplary embodiment, the expansion element may be expanded inside a portion (e.g. a limb) of a crimped stent device and subsequently contracted prior to or during removal of said expansion element.

With reference to FIG. 1E, in some embodiments, the low-profile expansion element 140 can be coupled to a hollow wire 142 that can be advanced over the contralateral wire 136 such that the low profile expansion element 140 can be moved through the second limb portion 126 by withdrawing the hollow wire 140 through the second puncture site. The low-profile expansion element 140 can have a diameter or cross-sectional size that is greater than an inner diameter or cross-sectional size of the crimped (also referred to as compressed) second limb portion 126 so that the second limb portion 126 is expanded as the low-profile expansion element 140 is moved through the second limb portion 126. Thereafter, with reference to FIG. 1F, the low-profile expansion element 140 can be completely withdrawn from the body and the second limb portion 126 can be in a partially expanded state that is sufficiently large to receive an expansion balloon or other mechanical expansion means therein, despite the external forces acting on the second limb portion 126. The expansion balloon or other mechanical expansion means can then be advanced over the contralateral wire 136 through the contralateral introducer sheath and into the second limb portion 126 to complete the expansion of the second limb portion 126. In some embodiments, the expansion balloon can be pulled through the ipsilateral introducer sheath and down into the second limb portion 126 to complete the expansion of the second limb portion 126. In some embodiments, the stent device 120 can be positioned such that the legs of the stent graft are pulled down to seat the graft onto the bifurcation of the aorta before any expansion is performed. In other embodiments, the stent device 120 can be positioned such that the legs of the stent graft are pulled down to seat the graft onto the bifurcation of the aorta as the stent 120 is being partially or fully expanded, or after the stent 120 is partially or fully expanded.

While the figures and description describe using the low-profile expansion element 140 to expand the second limb portion 126, in other embodiments, the low-profile expansion element 140 can be used to expand other portions of the stent device 120, including the main body portion 122 and/or the first limb portion 124. In some embodiments, the low-profile expansion element 140 can have an angled or beveled proximal end portion 142 to facilitate the movement of the low profile expansion element 140 through the second limb portion 126 and to cause the second limb portion 126 to more gradually expand as the low profile expansion element 140 is moved through the second limb portion 126. In some embodiments, the low profile expansion element 140 can have an extended tapered portion that can be one half or more of the length of the low profile expansion element 140, or from one-quarter to three-quarters of the length of the low profile expansion element 140, or from one-quarter to the entire length of the low profile expansion element 140, or of any value or any ranges of values within the foregoing ranges. In some embodiments, the low profile expansion element 140 can have a diameter greater than approximately 0.05 inch; less than approximately 0.25 inch; between approximately 0.05 inch and approximately 0.25 inch; between approximately 0.10 inch and approximately 0.25 inch; between approximately 0.10 and approximately 0.20 inch; or between approximately 0.10 inch and approximately 0.125 inch; including all values (e.g. decimal values) within the foregoing ranges. In exemplary embodiments, the expansion element has an initial (non-expanded) diameter of greater than approximately 0.05 inch, greater than approximately 0.10 inch, greater than approximately 0.175 inch, greater than approximately 0.20 inch or greater than approximately 0.25 inch before expansion including all values (e.g. decimal values) within the foregoing ranges. In exemplary embodiments, the expansion element has an expanded diameter of less than approximately 0.05 inch, less than approximately 0.10 inch, less than approximately 0.175 inch, less than approximately 0.20 inch or less than approximately 0.25 after expansion including all values (e.g. decimal values) within the foregoing ranges.

In some embodiments, the low profile expansion element 140 can be configured to expand the limb of the stent to be 30% of the final expanded diameter of the respective limb, or from 15% or approximately 15% or less to 40% or approximately 40% or more of the final expanded diameter of the respective limb, or from 20% or approximately 20% to 30% or approximately 30% of the final expanded diameter of the respective limb, or of any value or to and from any values within the foregoing ranges. In an exemplary embodiment, the contralateral limb has an inner diameter in the range of about 0.5-14 mm, including every decimal and integer value in this range. For example, the crimped contralateral limb may have an inner diameter of about 0.5-5 mm, preferably between about 1-3 mm. Following partial expansion with the expansion element, the contralateral limb may have an inner diameter of about 1-5 mm, preferably about 3-5 mm. With subsequent balloon expansion, the contralateral limb may have an inner diameter of about 5-14 mm, depending on the anatomy.

The exemplary embodiment disclosed herein can advantageously provide a streamlined surgical procedure. By maintaining the unibody bifurcated design, deficiencies of kissing stents and some versions of covered endovascular reconstruction of aortic bifurcation (CERAB) can be avoided, namely unequal blood flow division between the two limbs. Additionally, some embodiments of the balloon-expandable stents disclosed herein can be more beneficial to aortoiliac occlusive disease (AIOD) treatment, in part because some embodiments of the stent device 120 disclosed herein provide the physicians the ability to not only achieve higher radial strengths with smaller profiles, but to also provide the physicians with intermediary diameter/radial stiffness points, driven by the physician's controlled dilation.

Some embodiments of the system 100 can incorporate one or more components of the ENDOLOGIX AFX delivery system, such as a separate introducer to gain vascular access, a main handle and sheath for docking with the introducer, an inner core and atraumatic tip to transfer the system thru the introducer and anatomy, and/or a pre-cannulated contralateral limb wire to facilitate snaring and positioning the second limb portion 126 into the contralateral iliac artery, giving through and through access. Annexes A and B are parts of U.S. Pat. No. 8,808,350 which discloses some embodiments of the ENDOLOGIX AFX delivery system which as noted above can be incorporated into any embodiments of the system 100. The entire disclosure of U.S. Pat. No. 8,808,350 including the figures therefore forms part of the present disclosure including the specification and figures as set forth. In any embodiments disclosed herein, any components, features, or other details of the system 100, 200, 300 can have any of the components, features, or other details of any of the embodiments disclosed in Appendix A or be used according to any of the steps of any method embodiments disclosed in Appendix A in any combination with any of the components, features, or details of the system 100, 200, 300 or methods of use disclosed herein, as if such embodiments are explicitly disclosed herein.

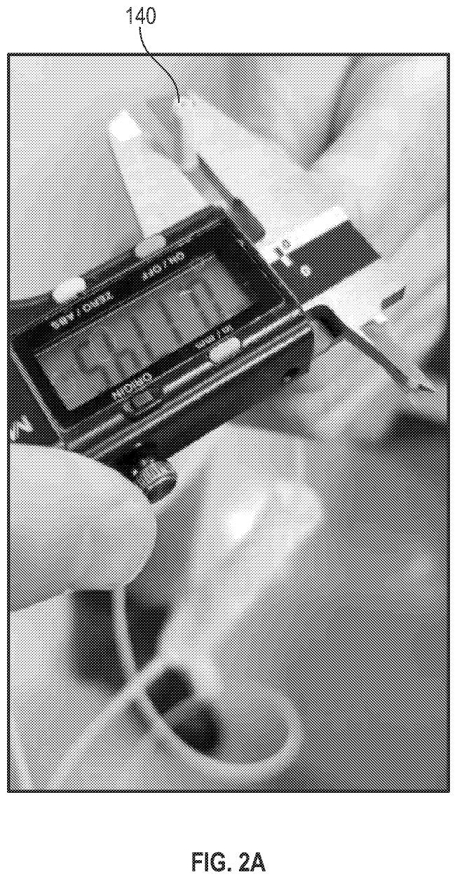

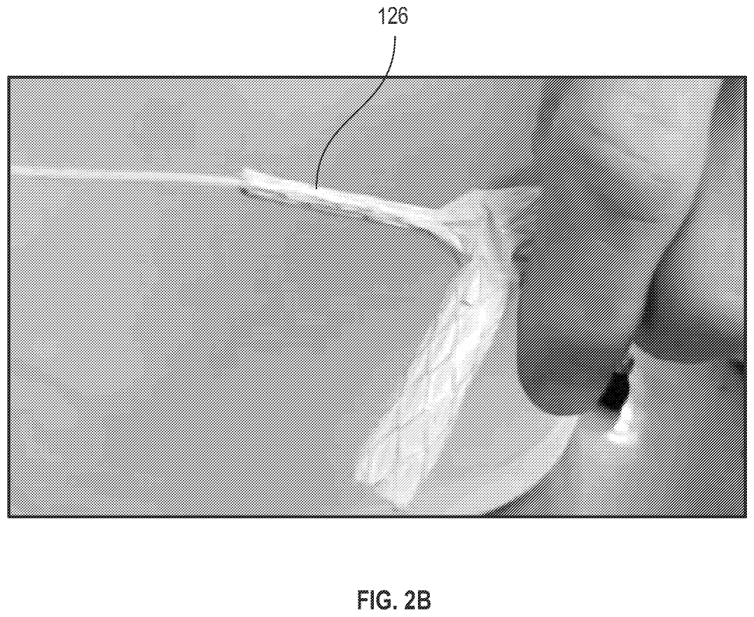

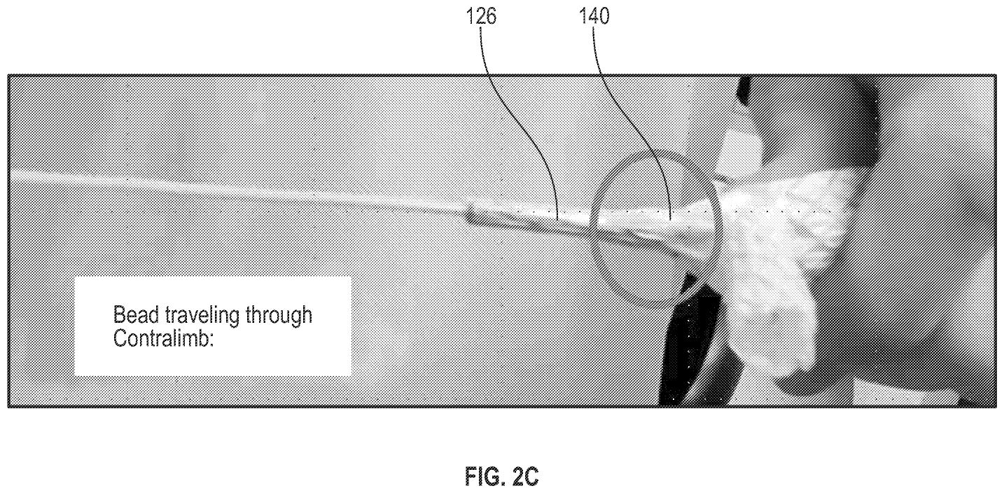

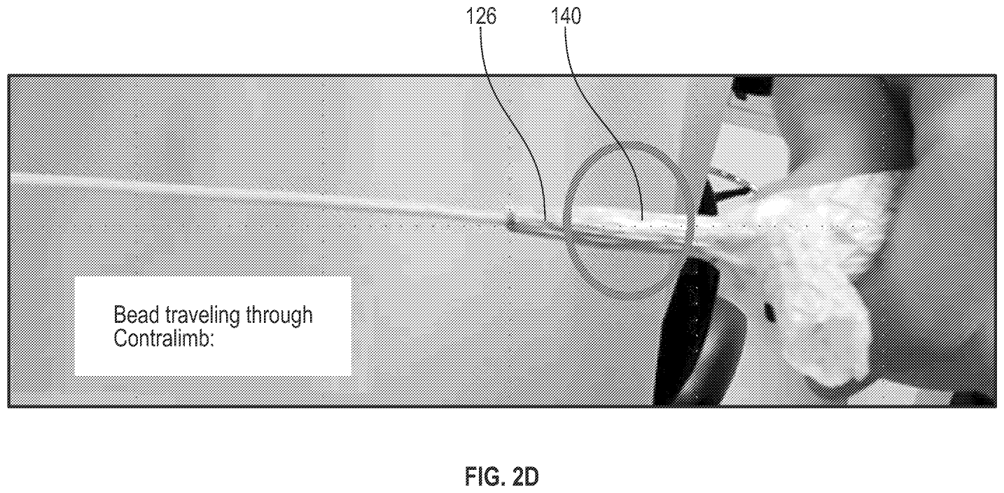

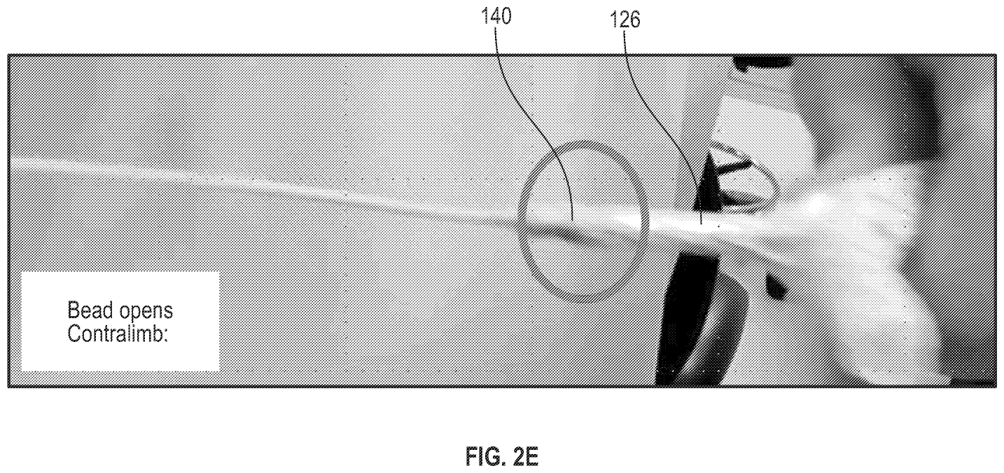

FIG. 2A shows an example embodiment of the low-profile expansion element 140. FIG. 2B shows an example embodiment of the second limb portion 126 of a stent in a crimped state. The second limb portion is also referred to herein as a crimped contralimb or compressed contralimb. FIGS. 2C-2E show an example embodiment of a second limb portion 126 of a stent in a crimped state, showing the expansion element 140 being moved through the second limb portion 126 and expanding the second limb portion 126 as the expansion element 140 is being moved through the second limb portion 126. The circle in each of FIGS. 2C-2E identifies the expansion element 140 in the second limb portion 126. In FIG. 2E, the expansion element 140 has moved through the entire length, or nearly the entire length, of the second limb portion 126 such that the entire length or nearly the entire length of the second limb portion 126 has been partially expanded by the expansion element 140.

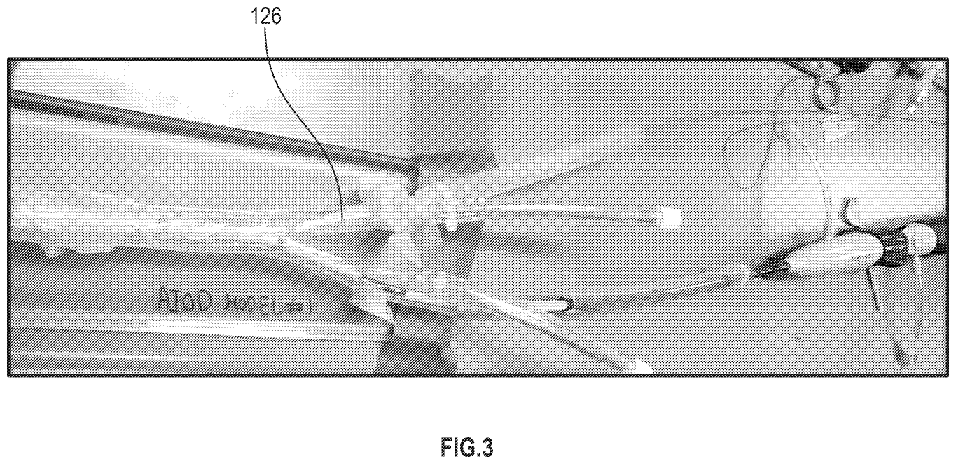

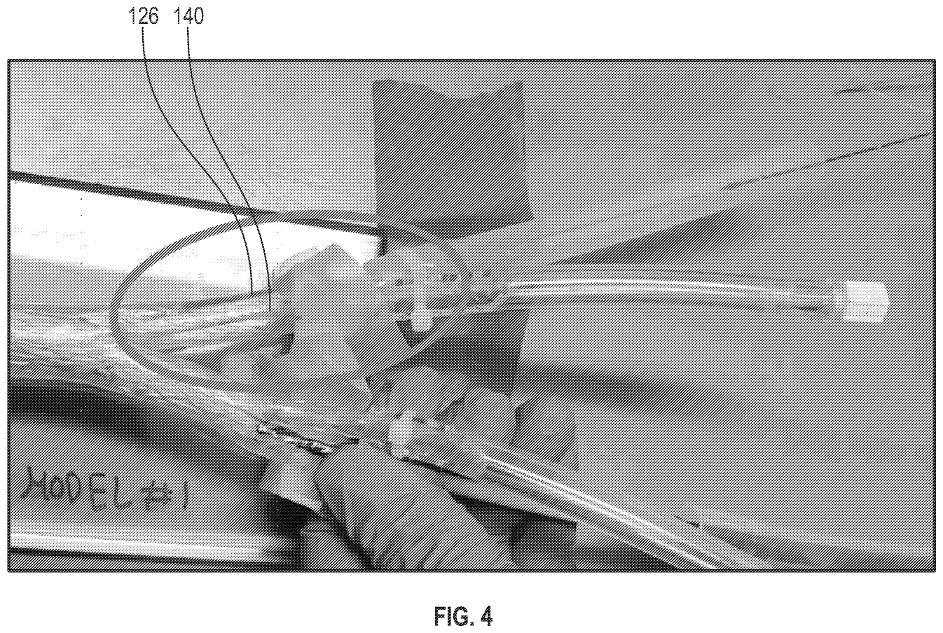

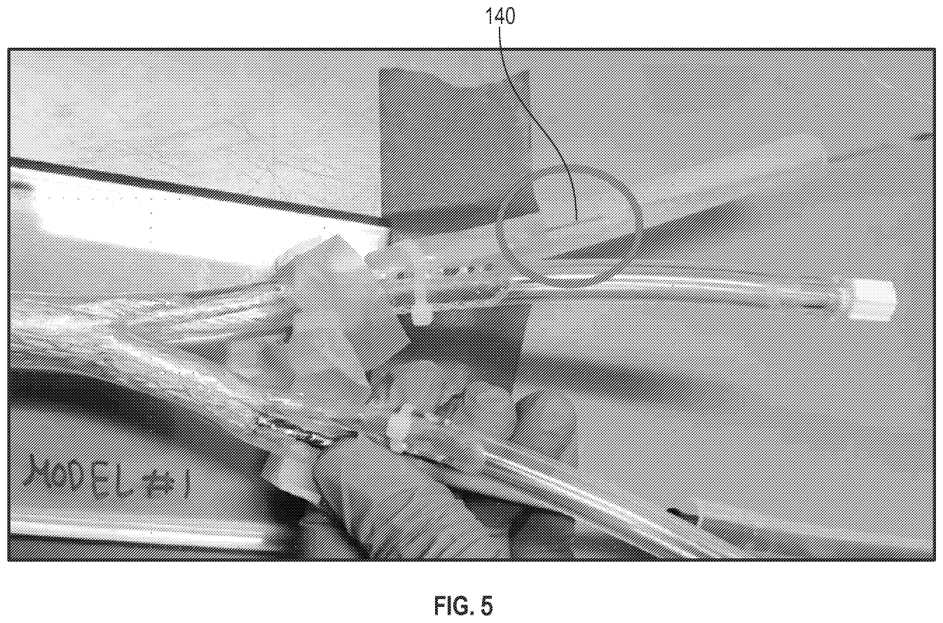

FIG. 3 shows an embodiment of a stent device in a crimped state, wherein the main body portion and the first limb portion of the stent device are being balloon expanded or are being prepared for balloon expansion. FIG. 4 shows the second limb portion of the stent device being partially expanded as the expansion element is moved through the second limb portion of the stent device. FIG. 5 shows the stent device after the expansion element has been pulled completely through the second limb portion of the stent device. The circle in each of FIGS. 4 and 5 identifies the expansion element 140 in the second limb portion 126.

Any other embodiments of the system 100 or stent 120 can have any of the components, features, or other details of any of the embodiments shown in FIGS. 3A-3C, in any combination with any of the other components, features, and/or other details of such embodiments.

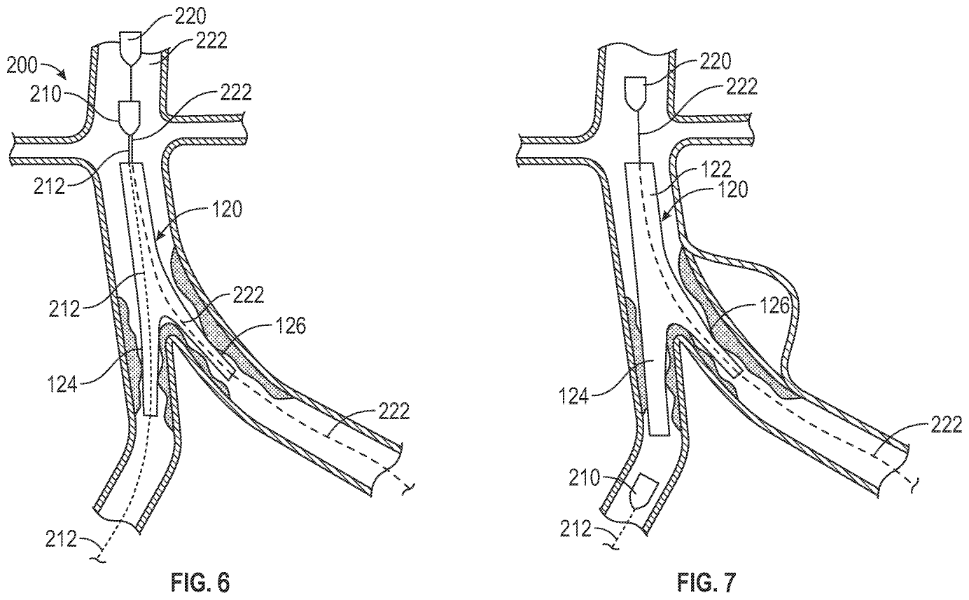

FIG. 6 shows another embodiment of a system 200 for treating aortoiliac occlusive lesions and other conditions at the aortic bifurcation, showing a bifurcated stent device 120 in a crimped or collapsed state. In any embodiments disclosed herein, any components, features, or other details of the system 200 can have any of the components, features, or other details of any other system embodiments disclosed herein or be used according to any of the steps of any other method embodiments disclosed herein, including without limitation any of the embodiments of the system 100 or methods of use thereof described herein, in any combination with any of the components, features, or details of the system 200 or methods of use disclosed herein. Similarly, any components, features, steps, or other details of any of the other system or method embodiments disclosed herein, including without limitation system 100 or methods of use thereof, can have any of the components, features, steps, or other details of any embodiments of the system 200 or methods of use thereof in any combination.

In some embodiments, the stent device 120 can be partially or fully expanded (or substantially fully expanded) by pulling or moving one or more, two or more, or three or more expansion elements through the stent device 120. In one example, with reference to FIG. 6, a first expansion element 210 can be used to expand the main body portion 122 and/or the first limb portion 124 of the stent device 120. The first expansion element 210 can be coupled with a first wire 212 that can be accessed through a first puncture side (for example, without limitation, through a first femoral puncture site-e.g., in the ipsilateral iliac artery). The first expansion element 210 can be moved through the main body portion 122 and the first limb portion 124 of the stent device 120 by withdrawing the first wire 212 through the main body portion 122 and the first limb portion 124 (e.g., through the first puncture site), thereby partially, fully, or substantially fully expanding the main body portion 122 and/or the first limb portion 124 of the stent device 120.

Similarly, a second expansion element 220 can be used to expand the second limb portion 126 of the stent device 120. The second expansion element 220 can be coupled with a second wire 222 that can be accessed through a second puncture side (for example, without limitation, in a second femoral puncture site-e.g., in the contralateral iliac artery). The second expansion element 220 can be moved through the main body portion 122 and the second limb portion 126 of the stent device 120 by withdrawing the second wire 222 through the main body portion 122 and the second limb portion 124 (e.g., through the second puncture site), thereby partially, fully, or substantially fully expanding the second limb portion 126 of the stent device 120. In some embodiments, though not required, the second expansion element 220 can be moved through the main body portion 122 and the second limb portion 126 of the stent device 120 after at least the main body portion 122 has been partially, fully, or substantially fully expanded. In some embodiments, though not required, the second expansion element 220 can be moved through the main body portion 122 and the second limb portion 126 of the stent device 120 after the main body portion 122 and the first limb portion 124 of the stent device have been partially, fully, or substantially fully expanded.

FIG. 7 shows the stent device 120 after the main body portion 122 and the first limb portion 122 of the stent device 120 have been partially expanded by the first expansion element 210. The expansion element 210 is removed along the first wire 212. Although not shown, the first wire, may remain within the stent device. Also as shown, the second limb portion 124 is still in a crimped state, with the second expansion element 220 being positioned distal to the stent device 120. In this state, the second expansion element 220 can then be moved through the main body portion 122 and the second limb portion 124 of the stent device 120 by withdrawing the second wire 222 to partially, fully, or substantially fully expand the second limb portion 126 of the stent device. If the main body portion 122, the first limb portion 124, and/or the second limb portion 126 are only partially expanded by the expansion elements 210, 220, the main body portion 122, the first limb portion 124, and/or the second limb portion 126 can be fully expanded or substantially fully expanded using an expansion balloon or any other suitable expansion device at any step in the process. The first and second expansion elements 210, 220 can be withdrawn through the first and second puncture sites, respectively.

Again, as with any other embodiments disclosed herein, the first and/or second expansion elements 210, 220 can have any suitable or desired shape, size, or other details. For example, and without limitation, the first and/or second expansion elements 210, 220 can have a tubular or cylindrical shape, a tapered cylindrical shape like that of a bullet, the shape of a bead, or otherwise.

In some embodiments, the first and/or second expansion elements 210, 220 (and/or any other expansion elements) can be positioned within or coupled with the delivery catheter (not shown) distal to main body portion 122 of the stent device 120 when the stent device is crimped onto the delivery catheter. This can, in some instances, decrease an overall profile size of the delivery device (e.g., when the stent device is in the crimped state on the delivery device). In some embodiments, the second expansion element 220 can be positioned distal to the first expansion element 210 and adjacent to the first expansion element 210, spaced apart from the first expansion element 210, or slightly overlapping the first expansion element 210. When the second expansion element 220 is positioned distal to the first expansion element 210 or adjacent to the first expansion element 210 without overlapping the first expansion element 210, the overall profile of the delivery device in the region of the expansion elements 210, 220 can be reduced.

In any embodiments disclosed herein, the delivery device can be configured to selectively support a distal end of the stent device 120 to inhibit the stent device from collapsing or substantially collapsing or substantially moving in an axial direction when the expansion element is moved through the stent device. In one embodiment, a lock mechanism such as a tether can be provided at the proximal region of the device connecting it with the delivery system wire and thereby preventing any potential collapse of the main body as the expansion element(s) travels towards the bifurcations.

In any embodiments disclosed herein, one or more of the expansion elements can be configured to be selectively expandable. For example, and without limitation, one or more of the expansion elements can have a removable sheath that can be configured to hold or maintain the respective expansion element in a collapsed or pre-expanded state. The removable sheath can be configured to be torn off of or otherwise removed from the expansion element. The expansion element can be configured to self-expand once the removable sheath has been removed. In some embodiments, the removable sheath can be coupled with a wire, such as a hollow wire, which can be used to at least withdraw the removable sheath from the expansion element and/or the body. In some embodiments, the removable sheath can be made from a perforated plastic shrink wrap. In some embodiments, the removable sheath can be configured to be removed from the expansion element by withdrawing a wire coupled with the removable sheath relative to the expansion element and/or a wire coupled with the expansion element. In some embodiments, the wire coupled with the expansion element can have sufficient stiffness or otherwise be configured to not buckle when the wire coupled with the removable sheath is withdrawn relative to the expansion element.

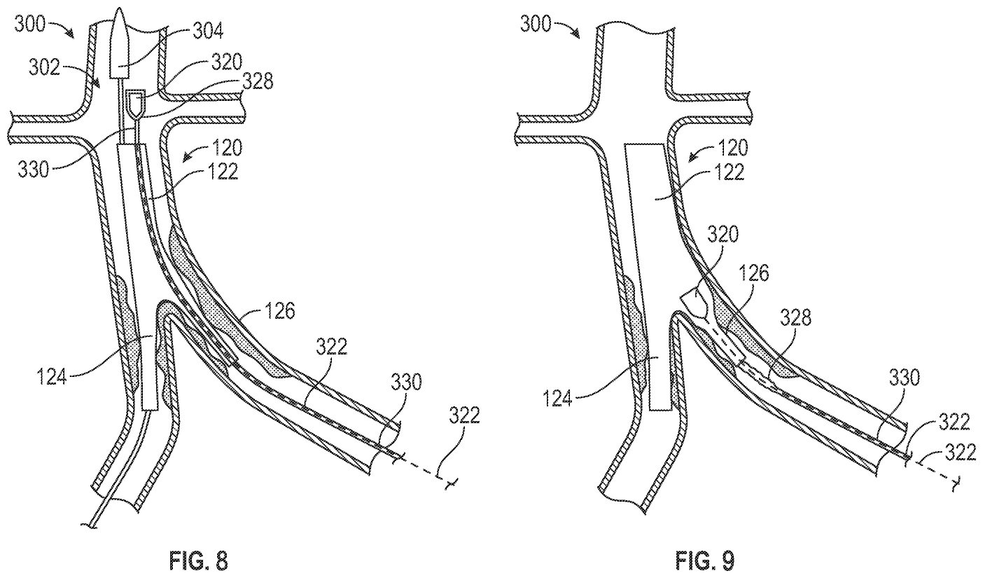

FIG. 8 shows another embodiment of a system 300 for treating aortoiliac occlusive lesions and other conditions at the aortic bifurcation, showing a bifurcated stent device 120 wherein the second limb portion 126 of the stent device 120 in a crimped or collapsed state. In any embodiments disclosed herein, any components, features, or other details of the system 300 can have any of the components, features, or other details of any other system embodiments disclosed herein or be used according to any of the steps of any other method embodiments disclosed herein, including without limitation any of the embodiments of the system 100, 200 or methods of use thereof described herein, in any combination with any of the components, features, or details of the system 300 or methods of use disclosed herein. Similarly, any components, features, steps, or other details of any of the other system or method embodiments disclosed herein, including without limitation system 100, 200 or methods of use thereof, can have any of the components, features, steps, or other details of any embodiments of the system 300 or methods of use thereof in any combination.

In some embodiments, the system 300 can have a delivery catheter 302 having a distal tip 304 and any of the other features of any of the other delivery catheter embodiments disclosed herein or used for deployment of bifurcated stents. In some embodiments, the stent device 120 can be partially or fully expanded (or substantially fully expanded) by pulling or moving one or more, two or more, or three or more expansion elements through the stent device 120. In some embodiments, the stent device 120 can be partially or fully expanded (or substantially fully expanded) by pulling or moving an expansion element having multiple portions (e.g., two or more, or three or more) of increasing diameter through the stent device 120. In any embodiments disclosed herein, as shown in FIG. 8, an expansion element 320 can be positioned proximal to the distal tip 302 of the delivery catheter. The expansion element can be positioned adjacent to a proximal end portion of the distal tip in any embodiments disclosed herein. The expansion element 320 can be used to expand the second limb portion 126 of the stent device 120. The expansion element 320 can be coupled with a wire 322. The wire 322 can be solid or can be hollow, sized and configured to pass over a guidewire. The expansion element 320 can be self-expanding and can be supported in a collapsed or reduced size state by a removable sheath 328. The removable sheath 328 can be coupled with a wire 330, that can be a hollow wire configured to pass over the wire 322.

As mentioned, the expansion element 320 can be coupled with a wire 322 that can be accessed through a second puncture side (for example, without limitation, through a second femoral puncture site-e.g., in the contralateral iliac artery). The expansion element 320 can be moved through the main body portion 122 and into the second limb portion 124 of the stent device 120 by withdrawing the wire 322 through the main body portion 122 and the second limb portion 126 (e.g., through the second puncture site) and positioned in a distal end portion of the second limb portion 126 of the stent device 120. Thereafter, the removable sheath 328 can be removed from the expansion element 320, so that the expansion element 320 can self-expand to a second state of the expansion element 320, in which the expansion element 320 has an increased size as compared to a first state of the expansion element (i.e., when the expansion element is constrained by the removable sheath). FIG. 9 shows the expansion element 320 after the expansion element 320 has been moved into the second limb portion 126 of the stent device 126 and expanded to the second state of the expansion element 320 by removing the removable sheath 328.

The removable sheath 328 can be removed from the expansion element 320 and from the body by withdrawing the wire 330 coupled with the removable sheath 328. Thereafter, the expansion element 320 can be moved through the second limb 126 of the stent device 120 by withdrawing the wire 322 coupled with the expansion element 320 through the second puncture site. In any embodiments herein, the second limb portion 126 of the stent device 120 can be partially, fully, or substantially fully expanded by the expansion element 320. An expansion balloon or other expansion device can thereafter be advanced through the second puncture site up through the second limb portion 126 of the stent 120 to further expand the second limb portion 126.

In some embodiments, the expansion element can be 100% larger (i.e., double the size), or approximately 100% larger, in a radial direction, perpendicular to a centerline axis of the expansion element, when the expansion element is in a second state as compared to when the expansion element is in the first state. In some embodiments, the expansion element can be from 50% larger or approximately 50% larger to 200% larger, approximately 200% larger, or more than 200% larger, in the radial direction when the expansion element is in a second state as compared to when the expansion element is in the first state, or from 75% larger or approximately 75% larger to 150% larger or approximately 150% larger in the radial direction when the expansion element is in a second state as compared to when the expansion element is in the first state, or of any value or range of values in any of the foregoing ranges.

### Other Details

In any embodiments disclosed herein, the stent device 120 can be a non-bifurcated stent wherein the expansion element can be used to partially, fully, or substantially fully expand all or a portion of the non-bifurcated stent.

In any embodiments disclosed herein, one or more, two or more, three or more expansion elements can be preloaded in the stent, or adjacent to the stent, within the delivery system, or otherwise coupled with the delivery system. For example, and without limitation, in any embodiments disclosed herein, the expansion element can be positioned at least partially within the second limb portion of the stent (e.g., adjacent to the bifurcation of the stent) when the stent is crimped to the delivery catheter. In any embodiments disclosed herein, the expansion element can be positioned at least partially within the main body portion of the stent when the stent is crimped to the delivery catheter.

In any embodiments disclosed herein, any portion of the stent (including the embodiments of the stent 120 disclosed herein) can be self-expanding. For example and without limitation, in some embodiments, the stent device 120 can be configured such that the main body portion 122, the first limb portion 124, and/or the second limb portion 126 can be self-expanding while the other(s) of the main body portion 122, the first limb portion 124, and the second limb portion 126 can be balloon expandable or otherwise mechanically expandable. For example, and without limitation, in some embodiments, the main body portion 122 and the first limb portion 124 of any embodiments disclosed herein can be self-expandable while the second limb portion 126 is balloon expandable. Alternatively, the main body portion 122 of any embodiments disclosed herein can be self-expandable while the first limb portion 124 and the second limb portion are balloon expandable. Any of the self-expandable portions can be secured within an outer sheath, can be secured in the collapsed state with a removable sheath, or otherwise.

In any embodiments disclosed herein, the stent device 120 or any portion thereof, the expansion devices (e.g., the balloons), and/or the expansion element 140 or other embodiments of the expansion elements disclosed herein can have radiopaque markers, radiopaque coatings, or other features that have increased visibility in fluoroscopy. Further, in any embodiments disclosed herein, the expansion element can have a PTFE cover or coating or be made from PTFE.

In any embodiments disclosed herein, the stent can have one or more branch limbs, limb extensions, or otherwise in addition to the first and second limb portions disclosed herein, or openings for receiving branch limbs therethrough, such as for renal arteries, lumbar arteries, or otherwise.

In any embodiments disclosed herein, a portion of the stent (such as a distal portion of the main body portion 122 of the stent 120) can be removably coupled or tethered to a portion of the delivery catheter to at least inhibit (e.g., prevent) the stent from migrating and/or collapsing in an axial direction while the expansion element is being moved through the stent. Removable sutures or other selectively removable fastening elements can be coupled with, for example and without limitation, a distal end portion (i.e., the end portion closest to the heart) to at least inhibit (e.g., prevent) the stent from migrating and/or collapsing in an axial direction while the expansion element is being moved through the stent. In some embodiments, a proximal stent can be used to anchor or secure a distal end portion of the stent 120 to the patient's vasculature.

Some embodiments of the delivery system for the balloon expandable bifurcated stent graft can utilize an introducer to gain vascular access. In some embodiments, the main trunk and ipsilateral limb of stent graft can be mounted on an expandable inner core of a delivery catheter (with the balloon expandable inner core mounted within the lumen of the main trunk and ipsilateral limb of the stent graft). The delivery catheter with the stent graft can be advanced through the introducer. As mentioned, the distal end of the delivery catheter can include an atraumatic tip to transfer the system through the introducer and anatomy. A main handle of the delivery catheter can be configured for docking with the introducer.

The system and method described above uses a single expansion element 140 (referred to a “bead”). In modified embodiments, two or more beads can be used. In such an arrangement, one bead can open or partially expand the main body and ipsilateral limb, and second bead can be used to open or partially expand the contralateral limb. In such an arrangement, a balloon catheter is subsequently advanced into the main body and ipsilateral limb to fully open these portions. This arrangement would allow for a further reduction in the initial profile of the delivery catheter. In modified embodiments, two more beads of different maximum diameters and/or shapes can be used to open the second limb portion 126 (or other portions of the stent). For example, a first smaller overall diameter bead could be used to initially expand a portion of the stent and then a second larger overall diameter bead could be used to further expand a portion of the stent.

In some embodiments, the system 100 can have favorable specific advantages such as lower profiles, improved accuracy, and increased radial strength.

In some embodiments, one or more expansion balloons can be eliminated using a self-expanding bead. In any embodiments disclosed herein, the bead can be a self-expanding bead that can be achieved in a number of manners, such as self-expanding foam, open cell Polyurethane foam, flexible nitinol membranes, wire cage structure, metal mesh cage, or variations thereof. Some embodiments can have a tube in the contralimb, which can have the advantage of maintaining the inner lumen of the limb. The bead locking mechanism can be achieved in any of a number of different ways, such as a heat shrink coverage, or a suture held in place with a wire lock.

Another conceptual variation that can be included in any method embodiments disclosed herein is incorporating a cross-over lumen, so the contra wire is cannulated from the ipsilateral side and snared from contralateral side, which can in some embodiments provide a track for the bead to be fed by the physician post-cannulating from outside the patient's ipsilateral side, into the bifurcation, and out of the patient's contralateral side. In some embodiments, the bead can be replaced with a balloon assembly, which could be attached to a luer post exiting the patient's contra side.

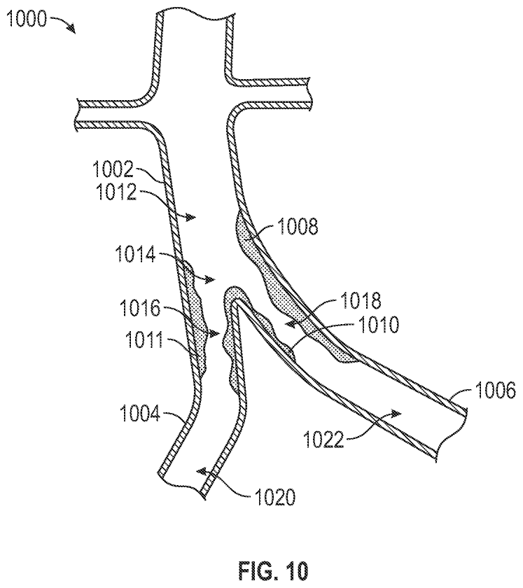

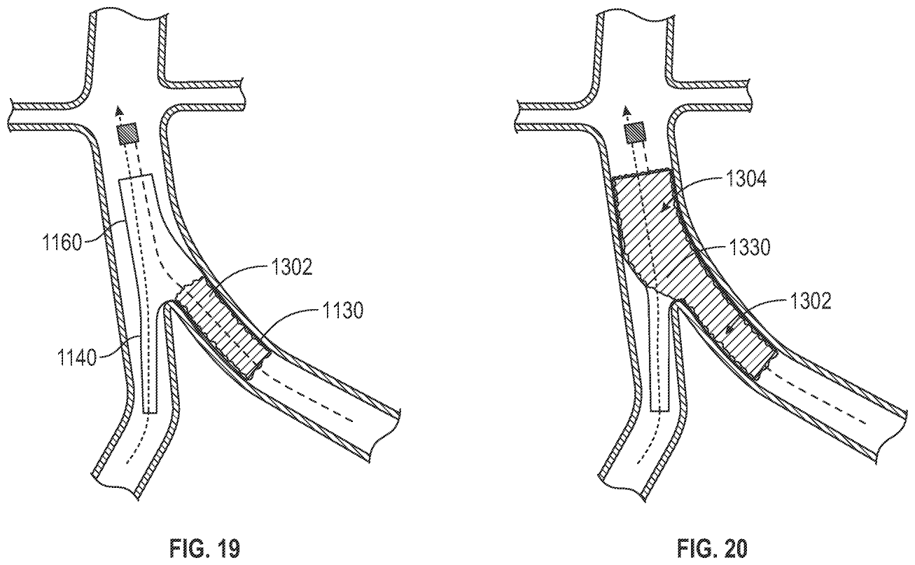

Certain embodiments of the present disclosure are further exemplified in FIGS. 10-27. Starting with FIG. 10, a bifurcated vessel 1000 has one or more occlusions 1008, 110 and 1011 in the main artery 1002 and branched arteries 1004, 1006 lumens. The occlusions result in an effectively reduced lumen sizes 1014, 1016 and 1018 in the main and branched arteries, respectively, compared to the non-occluded regions 1012, 1020 and 1022.

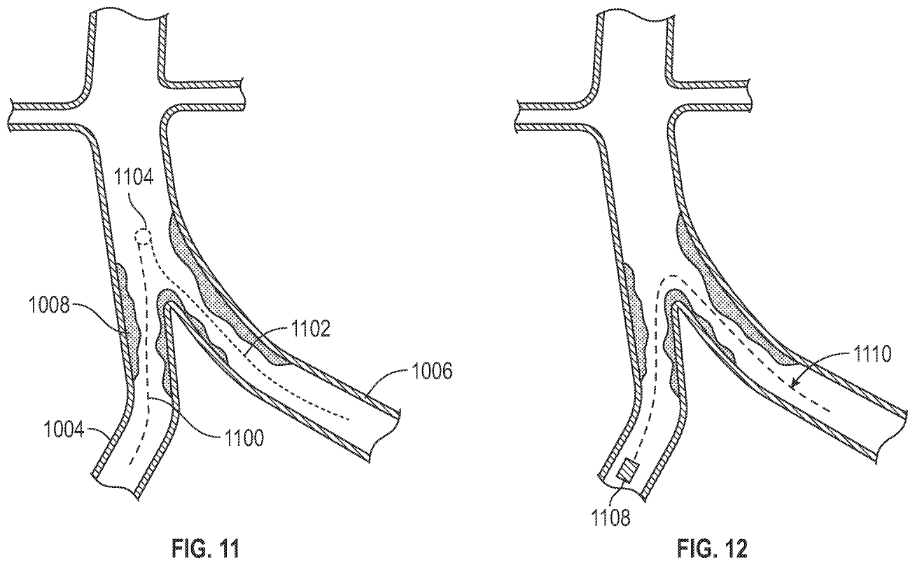

To address this condition an exemplary system and method shown in FIGS. 11 and 12 includes a contralateral guidewire (CW) 1100 delivered through the ipsilateral limb 1004 into the main artery 1002. The CW 1100 is captured with a snare 1104 located at the end of a snare wire 1102 that is inserted through the contralateral limb 1006. The snare wire 1102 is then pulled distally to move the CW 1100 into the contralateral limb 1006 and the delivery system 1108 into the main artery 1002. The delivery system may be positioned entirely or only partially within the occluded 1014 region of the main artery 1002.

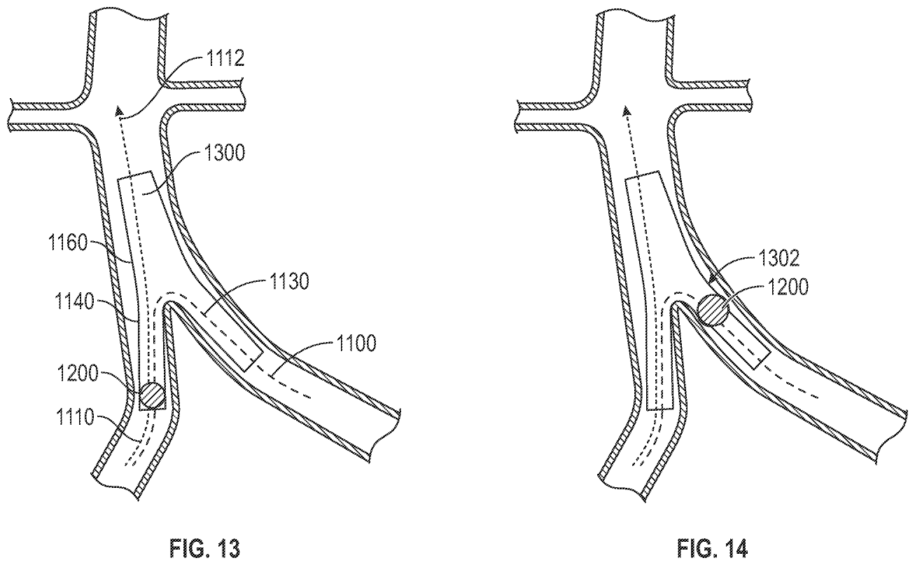

The remaining FIGS. 13-27 are depicted without the occlusions though it is understood that they are present in the main, branched or both lumens. Next, as shown in FIGS. 13, 14 and 15, the delivery system 1108 provides a bifurcated device 1300 (stent or stent-graft) seated on the bifurcation such that the ipsilateral limb 1140 is in a first vessel bifurcation and the contralateral limb 1130 is in a second bifurcation, with the main body 1160 being positioned in the main vessel lumen. Advantageously, an expansion element (e.g., bead) 1200 is pre-loaded in the ipsilateral limb 1140 of the device in a predeployment state and configured to travel over the CW 1100 resulting in the expanded 1302 contralateral limb. Of course, the bead 1200 may also expand the ipsilateral limb 1140 as well, prior to entering the contralateral limb. The contralateral limb expansion may be partial or full with respect to the vessel bifurcation lumen. The expansion element may be pre-loaded outside of the ipsilateral limb, inside at the proximal end, as well as at or near the device bifurcation.

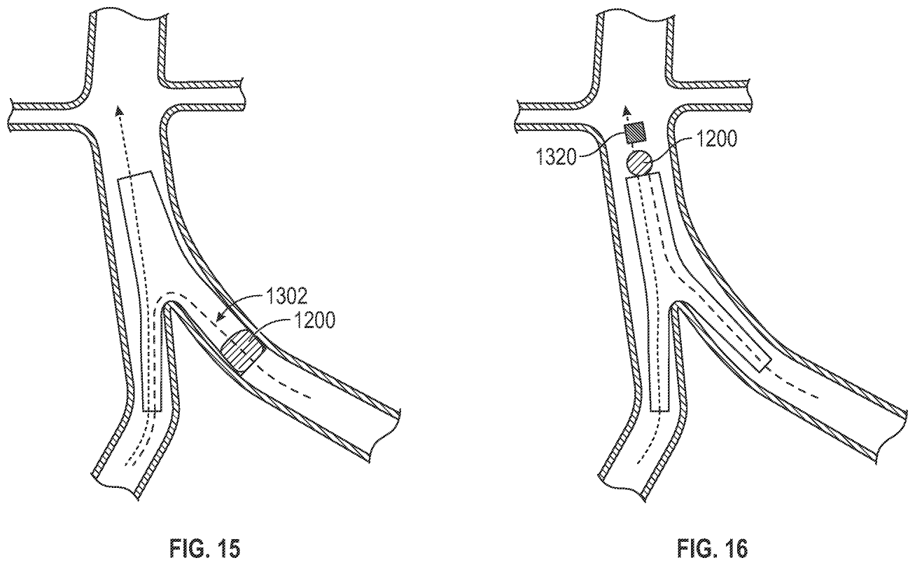

Also shown, the delivery system guidewire (GW) 1110 is positioned in the device 1300 and extends out of the main body 1160 with the delivery device tip 1112. As noted in this disclosure, the expansion element may be self-expanding. To that end, FIG. 15 illustrates an expansion element 1200 positioned in the contralateral limb having an increased diameter relative to its initial pre-loaded state, resulting in a greater contralateral limb expansion 1302.

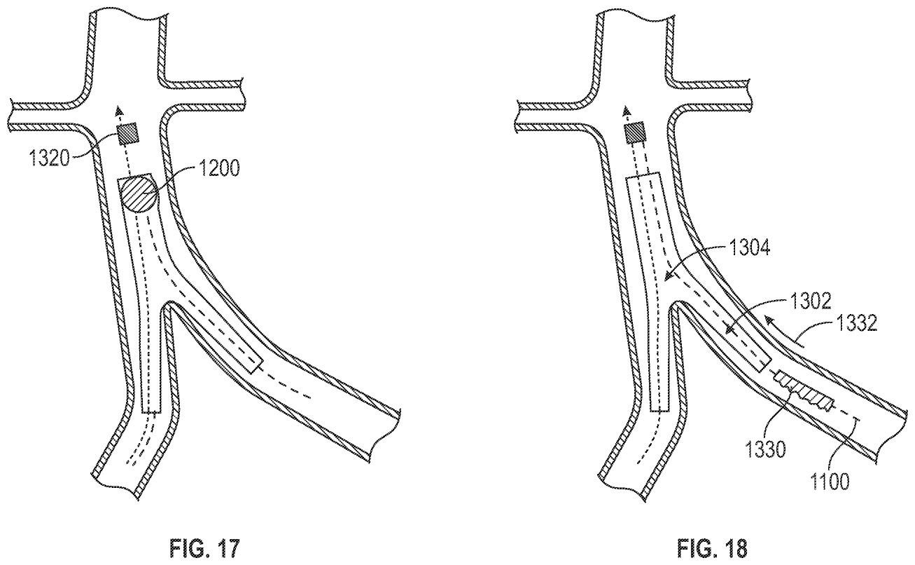

Alternatively, the expansion element 1200 may be pre-loaded at or near the proximal end of the device 1300 main body as shown in FIG. 16. Here, the CW and GW are locked together via the locking mechanism 1320. The locking mechanism can include a GW lumen and a CW lumen, where the CW is locked in the lumen along with a lock wire which releasably retains the CW. The CW may include any raised, tapered or otherwise altered feature to ensure secure locking within the lumen. As shown in FIG. 17, the expansion element 1200 travels long the CW 1100 and eventually out of the contralateral limb 1130 to expand the same. Here too the expansion element 1200 may expand the main body 1160 of the device 1300 as well.

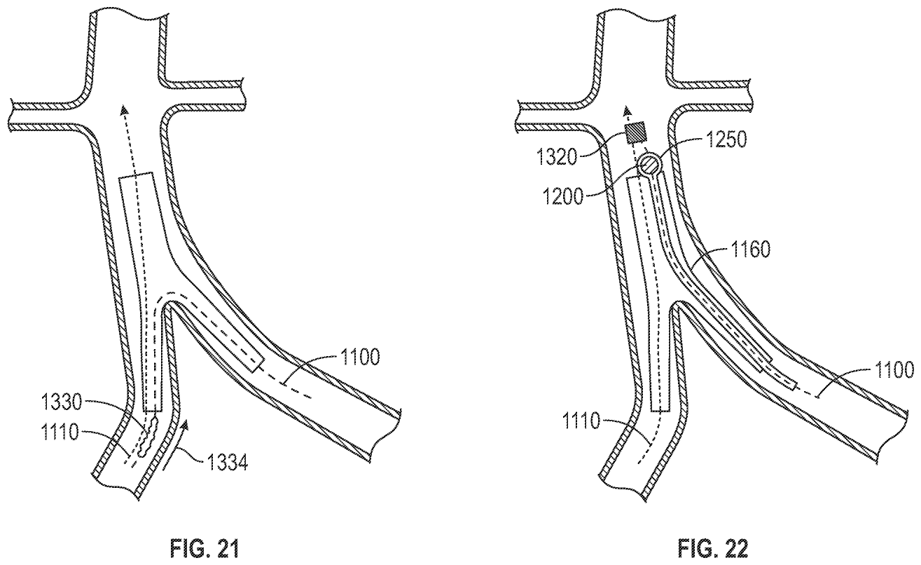

Following partial expansion of the contralateral limb 1130, a balloon 1330 is directed along the CW 1100 into the limb 1130 along the direction 1332, as depicted in FIG. 18. Of course the balloon may be inserted from the ipsilateral limb as depicted in FIG. 21 when employing the device and methodology of FIGS. 13-15. Inflating the balloon as shown in FIG. 18, results in the fully expanded contralateral limb 1302, eventually increasing the vessel diameter. The balloon 1330 can essentially take on any shape or size that is appropriate for fully expanding the limb as well as the main body. As such, the balloon length may span the entire contralateral limb 1130 or the entire length of the device (both contralateral limb and main body). Moreover, the balloon 1330 may comprise one or more cross sectional sizes along its length. As a non-limiting example, the balloon may be a stepped balloon. For instance, FIG. 20 illustrates a balloon that spans the length of the device and has a greater cross-section at the proximal region such that when inflated, both the main body and contralateral limb are expanded (1304 and 1302).

It is desirable that the expansion element traverse through the device with minimal impedance. Accordingly, the expansion element may be encapsulated in a low friction lumen such as PTFE. Moreover, the length of the CW 1100 and the expansion element 1200 may be encapsulated in a lumen 1250 as depicted in FIG. 22. This feature is generically applicable to all exemplary embodiments including those presented in FIGS. 13-15.

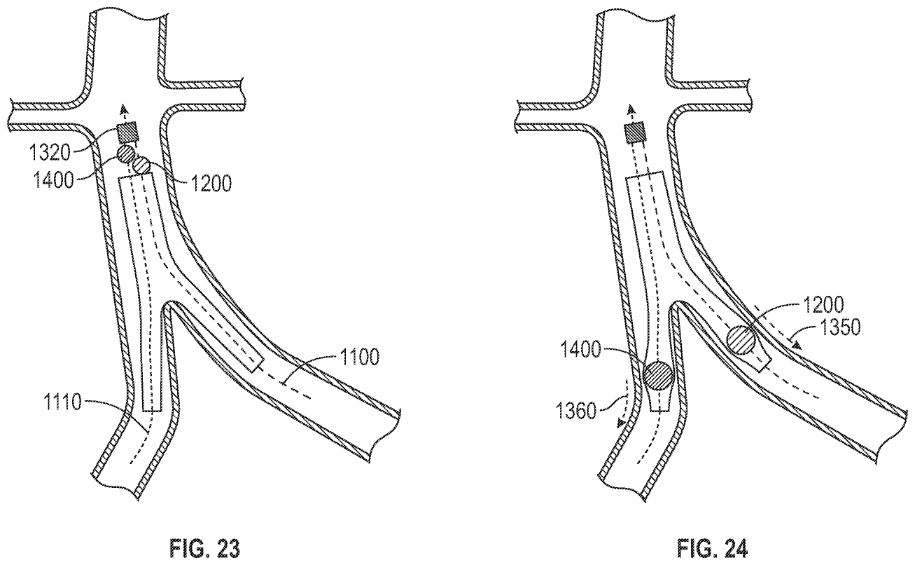

More than one expansion elements may be used in the exemplary systems and methods as shown in FIGS. 23 and 24. Here, a first expansion element 1200 and a second expansion element 1400 are pre-loaded in the delivery system at a proximal end of the device 1300. The first expansion element 1200 tracks over the CW 1100 and the second 1400 tracks over the GW 1110. Both wires are secured in the lock mechanism 1320. The relative positioning of the expansion elements 1200, 1400 may vary. Moreover, one or both may be placed inside the device and the shapes may be independent or complimentary to allow for a lower profile. Moreover, the order of translation may differ and the expansion elements 1200 and 1400 may move at different relative times in directions 1350 and 1360, respectively.

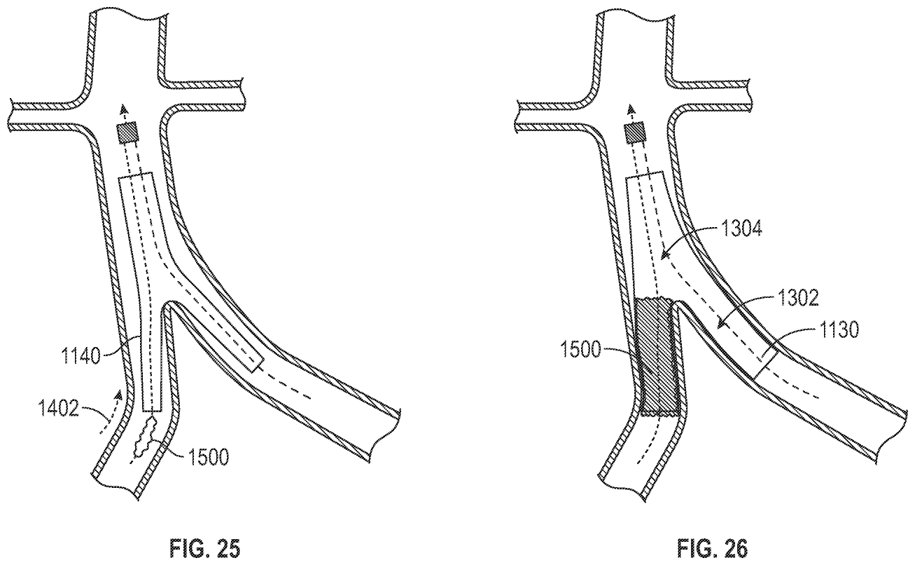

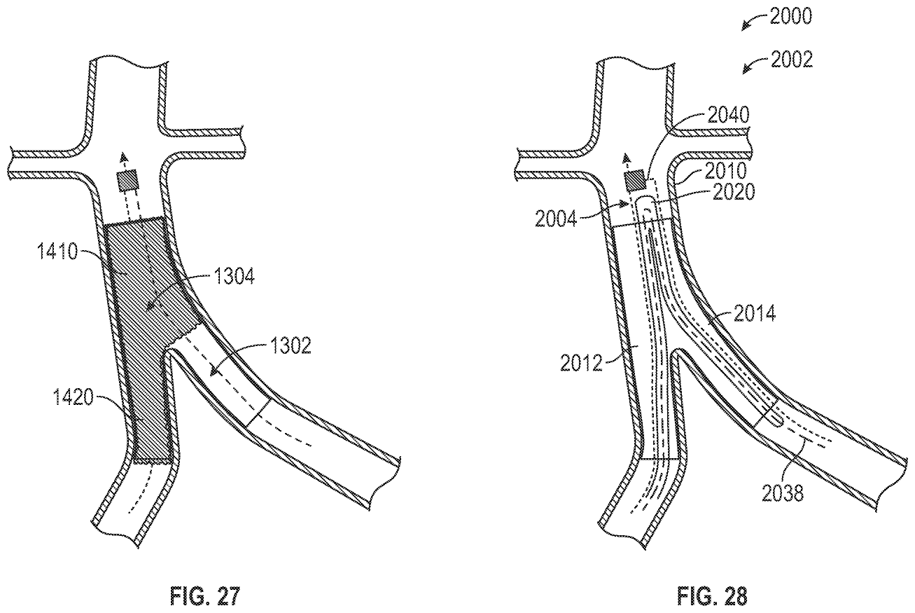

Once the contralateral limb 1130 is expanded an expansion balloon 1500 may be placed into the ipsilateral limb 1140 in the direction 1402 as shown in FIG. 25. As with the contralateral expansion balloon, the ipsilateral balloon 1500 may take on any shape and length desired for expanding the ipsilateral limb, the main body or both as shown in FIGS. 26 and 27. In particular, the balloon 1500 may be a stepped balloon where upon inflation expands the main body 1410 of the device and the ipsilateral limb 1420.

Following expansion of the main body and limbs of the device 1300, the delivery system may be removed leaving the bifurcated device in place, thereby improving effective diameter of the main vessel lumen and the branches.

FIG. 28 shows an embodiment of stent system 2000 comprising a stent 2002 (which can be a covered stent), or at least a portion thereof, and a stent deployment system 2004 used to deploy the stent 2002, or at least portion thereof. FIG. 28 shows the stent device 2002 positioned at an aortic bifurcation of a patient's vasculature.

In any embodiments, the system 2000 can be used for treating diseased vasculature in the body, including without limitation aortoiliac occlusive lesions and other conditions at the aortic bifurcation, iliac bifurcation, as well as other bifurcated and non-bifurcated arteries and vessels. Therefore, while certain embodiments are described as treating the aortic bifurcation, any embodiments of the systems, methods, and devices disclosed herein are configured to be, or can be configured to be, used in the treatment of any bifurcated and non-bifurcated vasculature in the body. Though not required, some embodiments of the stent 2002 can have a unibody design to avoid complications that are typically present with modular or multipart devices. Some embodiments of the methods disclosed herein include deploying a covered bifurcated stent device at the aortic bifurcation, wherein the stent is mechanically expandable (e.g., balloon expandable), is self-expanding, or is a hybrid mechanically expandable self-expanding device wherein some portions of the stent device (e.g., the main body portion, the first branch portion, and/or the second branch portion) are mechanically expandable and other portions of the stent device (e.g., the main body portion, the first branch portion, and/or the second branch portion that are not mechanically expandable) are self-expanding.

Any embodiments of the stent 2002 can have any of the components, features, or other details of any of the bifurcated stent embodiments disclosed herein in any combination with any of the components, features, and/or other details of the stent 2002. Any embodiments of the stent deployment system 2004 can have any of the components, features, or other details of any of the stent deployment system embodiments disclosed herein in any combination with any of the components, features, and/or other details of the stent deployment system 2004.

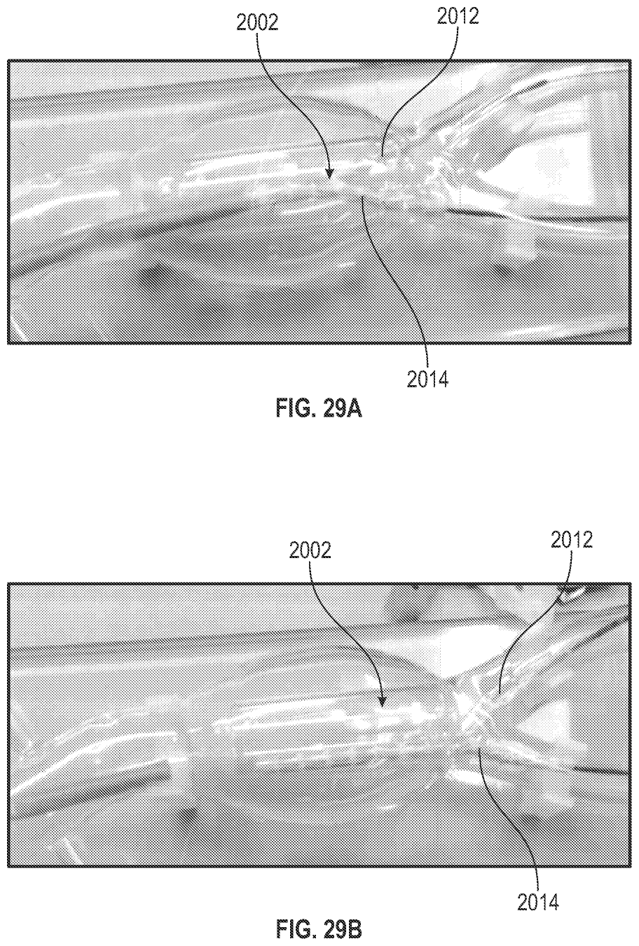

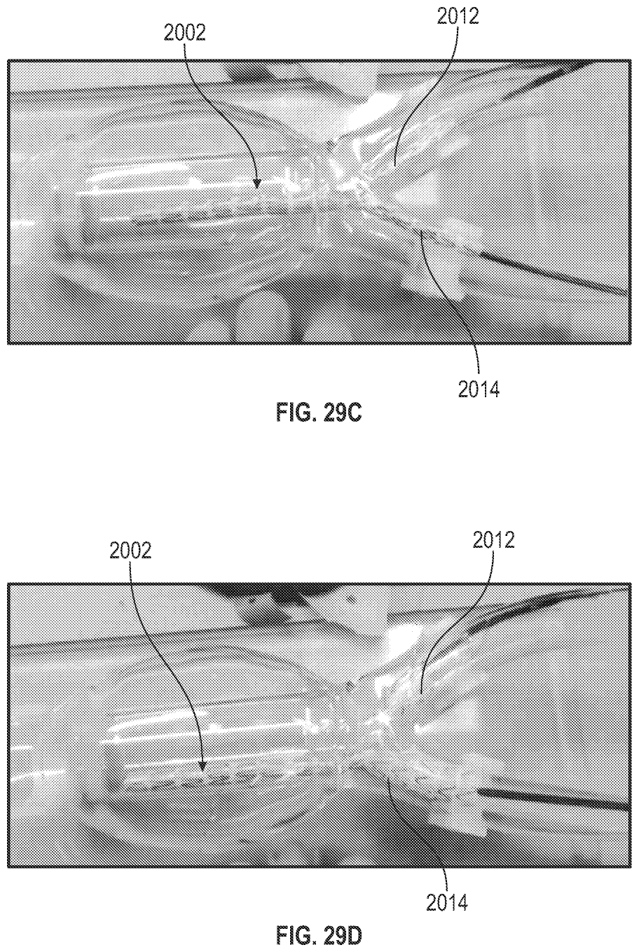

FIGS. 29A-29D show an embodiment of a deployment of at least portion of an embodiment of a stent 2002 in a bifurcation and an expansion of the first and second branch portions 2012, 2014 of the stent 2002. With reference to FIGS. 28-29D, some embodiments of the stent device 2002 can have a main body portion 2010 that is configured to extend into the aortic artery, a first downwardly extending limb portion 2012 configured to extend into a first iliac artery (e.g., the ipsilateral or the contralateral artery or limb) and a second downwardly extending limb portion 2014 (also referred to herein as the contralimb) configured to extend into a second iliac artery (e.g., the other of the ipsilateral and the contralateral artery or limb), as shown in FIG. 28.

Although the figures show an aneurytic aortic bifurcation, the use of the embodiments disclosed herein are not limited to use for treatment of abdominal aortic aneurysms. The embodiments of the devices disclosed herein can be used to treat a wide range of diseases and conditions of the aorta and aortic bifurcation, including without limitation aortoiliac occlusive disease (AIOD) and other aneurytic, embolic, and occluded aortic conditions. Some embodiments of the stent device 2002 disclosed herein can have the advantage of having a lower profile than some variations of conventional self-expanding stent devices, which can be advantageous when treating certain conditions, such as closed or partially closed aortic arteries.

Any embodiments of the stent device 2002 disclosed herein can be an uncovered mechanically expandable (e.g., balloon expandable) stent or a covered mechanically expandable (e.g., balloon expandable) stent of a unibody construction or other one-piece configuration wherein the main body portion 2010, the first downwardly extending leg portion 2012, and the second downwardly extending leg portion 2014 are connected together before deployment. In some embodiments, the main body portion 2010, the first downwardly extending leg portion 2012, and the second downwardly extending leg portion 2014 can be integrally formed. In some embodiments, the main body portion 2010, the first downwardly extending leg portion 2012, and the second downwardly extending leg portion 2014 can be separately formed and coupled together. In some embodiments, the main body portion 2010 and the first downwardly extending leg portion 2012 can be integrally formed and the second downwardly extending leg portion 2014 can be separately formed and coupled with the main body portion 2010 and the first downwardly extending leg portion 2012.

The frame 2014 of the stent 2002 can have any desired or suitable shape or configuration and can be made from laser cut tubing, wire, or by other known or later developed techniques and materials. In any embodiments, the frame 2014 of the stent device 2002 can be made from any suitable material, including stainless steel, cobalt chromium, or any other suitable metallic alloy or other material. In any embodiments, the graft can be made from any suitable material for grafts, including polyester, polyester/spandex, expanded polytetrafluoroethylene (ePTFE), or any other suitable or acceptable material. Some embodiments of the stent device 2002 can include a balloon expandable bifurcated stent structure that forms an inverted “Y” shape similar to a unibody stent graft structure. In such embodiments, the main body portion 2010 in the expanded configuration can have a larger external and internal diameter than the downwardly extending leg portions 2012, 2014.

Some embodiments of the system 2000 can be used to treat a diseased bifurcated vessel that can have one or more aneurysms, occlusions, or otherwise in the main artery and/or branched arteries. With reference to FIGS. 28-29D, in any embodiments disclosed herein, the delivery device 2004 can have an expansion balloon 2020 that can be preloaded in the stent 2002 and can be configured to extend across the bifurcation. For example and without limitation, in some embodiments, the expansion balloon 2020 can be configured to extend through all or a portion of the first branch portion 2012 of the stent 2002, past the bifurcation in the patient's vasculature and/or the bifurcation in the stent 2002, and through all or a portion of the second branch portion 2014 of the stent 2002.

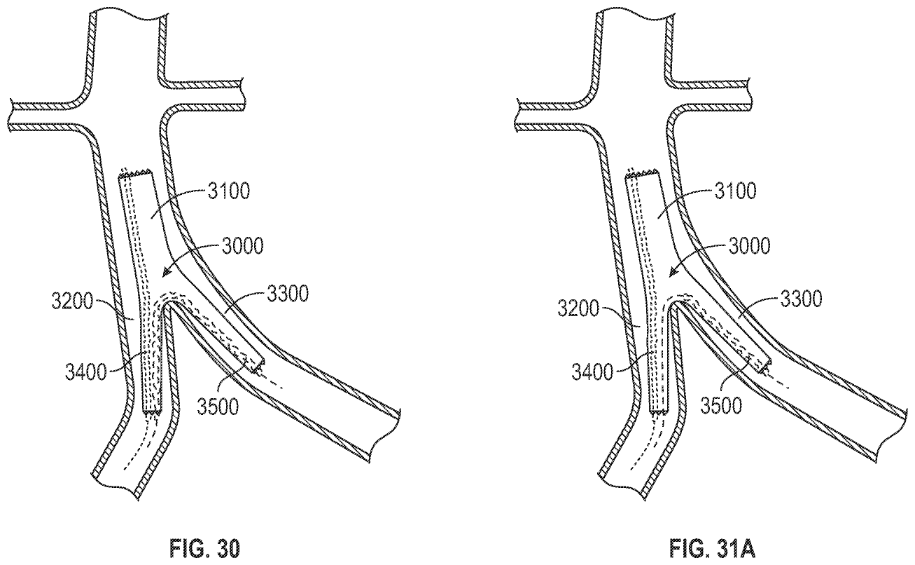

In some embodiments, the expansion balloon 2020 can be made from a flexible material that is configured to bend around the bifurcation in the stent 2002 when the expansion balloon 2020 is in the deflated state and when the expansion balloon 2020 is in the inflated state. In some embodiments, the expansion balloon 2020 can be configured to be biased to bend around the bifurcation in the stent 2002 (also exemplified in FIGS. 30-32B) at least when the expansion balloon 2020 is in the expanded state. For example and without limitation, in some embodiments, the expansion balloon 2020 can have a longer length along the side of the expansion balloon 2020 that is positioned further away from the center of the bend radius of the expansion balloon 2020 and/or further from the bifurcation. In some embodiments, a surface of the expansion balloon 2020 that is positioned further away from the center of the bend radius of the expansion balloon 2020 and/or further from the bifurcation can have corrugations to allow the expansion balloon 2020 to maintain a desired angle of the bend for the bifurcation when the expansion balloon 2020 is expanded. In some embodiments, a surface of the expansion balloon 2020 that is positioned further away from the center of the bend radius of the expansion balloon 2020 and/or further from the bifurcation can be more flexible than other portions of the expansion balloon 2020 to maintain a desired angle of the bend for the bifurcation when the expansion balloon 2020 is expanded. Some embodiments of the expansion balloon 2020 can have any combination of the foregoing features and all such combinations are specifically contemplated herein as if explicitly stated herein. In any embodiments disclosed herein, the balloon 2020 can be preloaded in the stent 2002.

As shown in FIG. 28, an inner shaft 2038 can extend through the expansion balloon 2020 in some embodiments. A contralateral guidewire 2040 can extend through the first and second branch portions 2012, 2014 to assist in positioning the second branch portion 2014 in the second branch (e.g., the contralateral branch) of the patient's vasculature. In some embodiments, the guidewire 2040 can be delivered through the ipsilateral limb into the main artery and be captured with a snare located at the end of a snare wire that is inserted through the contralateral limb. The snare wire can then be pulled distally to move the guidewire 2040 into the contralateral limb.

FIG. 29A shows an embodiment of the stent 2002 being deployed from a sheath 2026. However, an outer catheter sheath 2026 is not necessary for the system 2000. The stent 2002 as shown in FIG. 29A has already been positioned in the bifurcation of the patient's vasculature, with the main body of the stent 2002 positioned in the patient's aorta, the first branch portion 2012 of the stent 2002 positioned in a first branch of the patient's vasculature, and the second branch portion 2014 of the stent 2002 positioned in a second branch of the patient's vasculature. A balloon or at least a portion of a balloon 2020 extends through all or a portion of the first branch portion 2012 of the stent 2002, past the bifurcation in the patient's vasculature and/or the bifurcation in the stent 2002, and through all or a portion of the second branch portion 2014 of the stent 2002. FIG. 29C shows the stent 2002 before the balloon 2020 has been expanded. FIG. 29D shows the stent 2002 after the balloon 2020 has been expanded or at least partially expanded such that the first branch portion 2012 of the stent 2002 and the second branch portion 2014 of the stent 2002 are expanded with the balloon 2020.

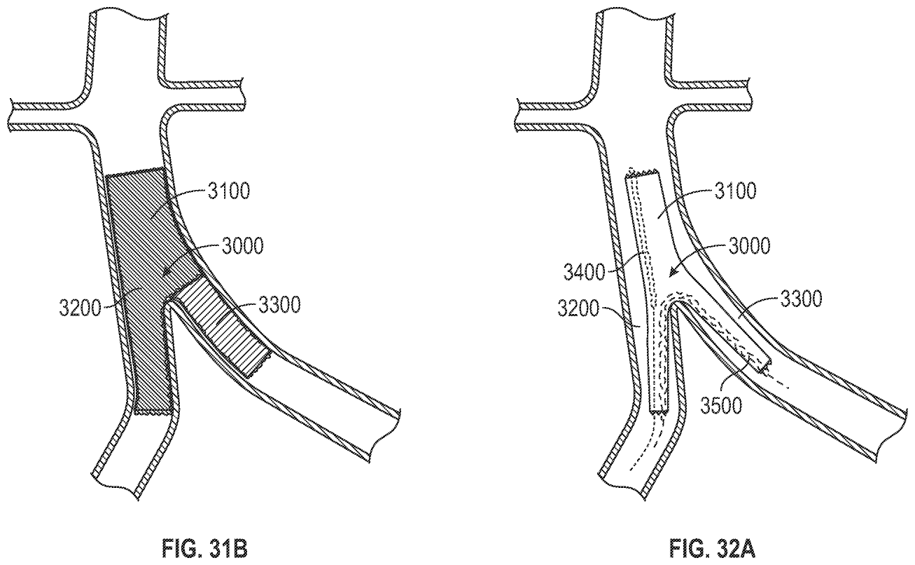

In some embodiments, the balloon 2020 can be configured to only expand the first and second branch portions 2012, 2014 of the stent 2002. In some embodiments, the balloon 2020 can be configured to extend also into the main body portion 2010 of the stent 2002 to expand the first and second branch portions 2012, 2014 of the stent 2002 and to simultaneously or approximately simultaneously expand the main body portion 2010 of the stent 2002. In some embodiments, with reference to FIG. 30, a second balloon 3500 can be positioned in the limbs or main body portion 3100 of the stent device 3000 and can be configured to be expanded simultaneously or approximately simultaneously with the first balloon 3400, or to be expanded after the first balloon 3400 is expanded, or before first balloon 3400 is expanded. Thereafter, the second balloon 3500 can be deflated and withdrawn through the first branch 3200 of the patient's vasculature or through the second branch 3300 of the patient's vasculature. In some embodiments, as shown in FIGS. 31A and B, a first balloon 3400 can extend through the first branch portion 3200 of the stent device 3000 and into the main body portion 3100 of the stent device, and a second balloon 3500 can be positioned in the second branch portion 3300 of the stent device 3000. The first balloon 3400 may be a stepped balloon such that it expands the main body 3100 and first branch 3200 as shown in FIG. 31B. As such, in combination with the expanded second balloon 3500, the entire stent device 3000 is expanded.

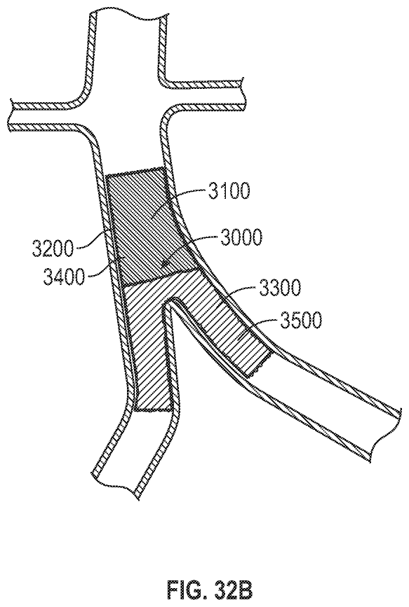

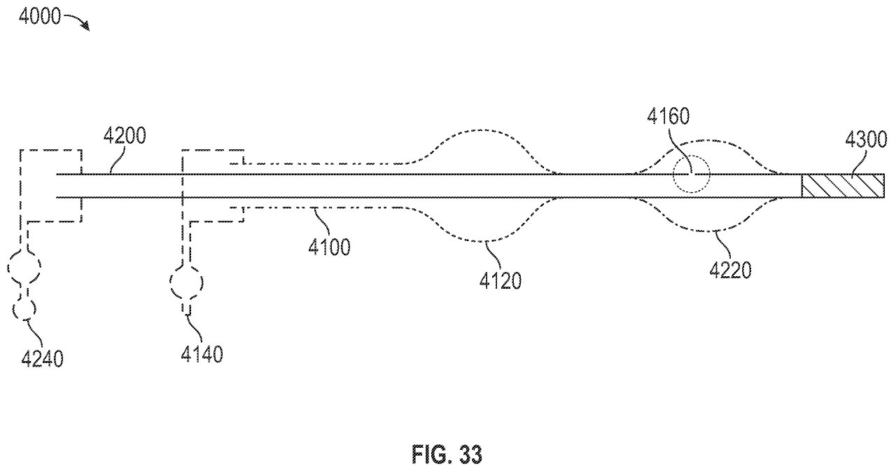

In some embodiments, the second balloon 3500 which extends through the first and second branch portions 3200, 3300 of the stent device 3000 can be more flexible than the first balloon 3400, if any, which extends through the main body portion of the stent device 3000. In the exemplary embodiment shown in FIGS. 32A and B, the second Balloon 3500 expands the first and second branches, 3200, 3300 while the first balloon 3400 expands the main body 3100 of the stent device 3000. FIG. 33 is an exemplary embodiment of a delivery system 4000 for inflating a first balloon 4120 and a second balloon 4220 via the first inflation port 4140 and second inflation port 4240, respectively. The first balloon 4120 is connected to and positioned with the outer shaft 4100 while the second balloon 4220 is connected to and positioned with the inner shaft 4200. The inner shaft 4200 can be slidably received within the outer shaft 4100 and have a closed end 4300. The second balloon 4220 can have an inflation port 4160 for expanding the balloon 4220 with inflation material. Although not shown, the first balloon will also have a port for inflation material.

Some embodiments of the system 2000 can be configured such that the stent 2002 is crimped in a compressed configuration about an inner core of the delivery catheter 2022. Some embodiments of the catheter 2022 can be advanced through a puncture site in a first iliac artery through an introducer and can be advanced past the bifurcation of the aorta, or advanced through any puncture site or opening into any portion of a patient's vasculature. The contralateral wire 2040 can be withdrawn through a second puncture site in a second of the iliac arteries. In some embodiments, the sheath 2026 can be withdrawn through the first puncture site, exposing the stent device 2002 which can then be moved toward the bifurcation with the first branch portion 2012 being withdrawn into the first iliac artery (e.g., without limitation, the ipsilateral iliac artery) and the second limb portion 2014 being moved or withdrawn into the second iliac artery (e.g., without limitation, the contralateral iliac artery). The stent device 2002 can still be in a contracted or low profile state while the stent is positioned in the patient's vasculature. In some embodiments, the stent 2002 can be withdrawn toward the aortic bifurcation so that the bifurcation portion of the stent 2002 is moved into contact with the aortic bifurcation, so as to seat the stent 2002 at the aortic bifurcation. The main body portion 2010 and the first branch portion 2012 can then be expanded, such as with an expansion balloon or other mechanically expandable device, or the main body portion and/or the first branch portion 2012 can be self-expanded if the stent is a hybrid stent.

FIG. 33 shows another embodiment of a stent deployment system. With reference to FIG. 33, an inflation lumen for each of two or more expansion balloons can be coaxially positioned. This can substantially reduce a profile of the device and simplify the delivery procedure. For example and without limitation, a first balloon (e.g., Balloon 1) can be coupled with a first shaft (e.g., an outer shaft) and a second balloon (e.g., Balloon 2) can be coupled with a second shaft (e.g., an inner shaft). The outer shaft can be positioned over and around an outside surface of the inner shaft such that the outer shaft is coaxial or approximately coaxial with the inner shaft. In some embodiments, the first balloon can be used to expand a first branch portion of a stent and the second balloon can be used to expand a second branch portion of the stent or the main body portion of the stent.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A device for treating a patient, comprising:
a delivery catheter;
a compressed expandable bifurcated stent having a main body portion, a first limb portion, and a second limb portion; and
an expansion element pre-loaded in a portion of the stent or delivery device.

2. The device of claim 1, further comprising an expansion balloon pre-loaded in at least the main body portion and the first limb portion such that the main body portion and the first limb portion are crimped around the expansion balloon in a predeployment state.

3. The device of claim 1 or 2, wherein the expansion element is configured to partially expand the second limb portion of the stent as the expansion element is pulled through the second limb portion of the stent.

4. The device of any one of claims 2-3, wherein the wherein the expansion balloon is a stepped balloon, wherein a distal portion of the stepped balloon is positioned within the main body portion of has a larger expanded diameter than a more proximal portion of the expansion balloon which is positioned within the first limb portion.

5. The device of any one of claims 1-4, wherein the compressed expandable bifurcated stent includes a graft covering.

6. The device of any one of claims 1-5, herein the expansion element is coupled to a hollow wire.

7. The device of claim 6, wherein the hollow wire is positioned over a guidewire.

8. The device of any one of claims 1-7, wherein the expansion element includes a tapered portion.

9. The device of any one of claims 1-8, wherein the expansion element is self-expanding.

10. The device of claim 9, wherein the expansion element is expandable from a first state to a second state, wherein the expansion element is larger in a radial direction when the expansion element is in the second state.

11. The device of any one of claims 9-10, wherein the expansion element is covered by a removable sheath that is coupled with a wire.

12. The device of any one of claims 1-11, further comprising a second expansion element, wherein the second expansion element is configured to expand at least a first limb portion of the stent.

13. The device of any one of claims 1-12, wherein the device is configured for treating the infrarenal aorta and iliac arteries of the patient.

14. The device of any one of claims 1-13 wherein the compressed expandable bifurcated stent is balloon expandable.

15. A method for treating a patient, comprising:
advancing a delivery catheter into a patient's aorta through a femoral artery puncture site to advance an expandable bifurcated stent having a main body portion, a first limb portion, and a second limb portion into the patient's aorta;
expanding the main body portion and the first limb portion of the stent; and
moving an expansion element through the second limb portion of the stent to partially expand the second portion of the stent.

16. The method of claim 15, comprising further expanding the second limb portion of the stent using an expansion balloon.

17. The method of any one of claims 15-16, wherein the method comprises treating the patient's infrarenal aorta and iliac arteries.

18. The method of any one of claims 15-17, wherein the expansion element is positioned within the delivery catheter when the delivery catheter is advanced into the patient's aorta.

19. The method of any one of claims 15-18, wherein expanding the main body portion and the first limb portion of the stent comprising using an expansion balloon with a stepped balloon with a larger and smaller diameter portions.

20. The method of any one of claims 15-19, wherein moving an expansion element through the second limb portion of the stent to partially expand the second portion of the stent; comprises pulling on a hollow wire coupled to the expansion element.

21. The method of claim 20, wherein the hollow wire is pulled over a guidewire.

22. The method of any one of claims 15-21, comprising moving a first expansion element through at least the first limb portion of the stent to at least partially expand the first limb portion of the stent before moving the expansion element through the second limb portion of the stent to partially expand the second portion of the stent.

23. A device for treating the infrarenal aorta and iliac arteries, comprising:
a delivery catheter;
a compressed balloon expandable bifurcated stent having a main body portion, a first limb portion, and a second limb portion;
an expansion element pre-loaded in a portion of the stent; and
an expansion balloon pre-loaded in at least the main body portion and the first limb portion such that the main body portion and the first limb portion are crimped around the expansion balloon in a predeployment state;
wherein:
the expansion element is configured to partially expand the second limb portion of the stent as the expansion element is pulled through the second limb portion of the stent.

24. A method for treating the infrarenal aorta and iliac arteries, comprising:
advancing a delivery catheter into a patient's aorta through a femoral artery puncture site to advance a balloon expandable bifurcated stent having a main body portion, a first limb portion, and a second limb portion into the patient's aorta;
expanding the main body portion and the first limb portion of the stent using an expansion balloon;
moving an expansion element through the second limb portion of the stent to partially expand the second portion of the stent; and
further expanding the second limb portion of the stent using a second expansion balloon.

25. A device for treating a patient, comprising:
a delivery catheter;
a compressed expandable bifurcated stent having a main body portion, a first limb portion, and a second limb portion; and
an expansion element pre-loaded in at least a portion of the stent or delivery device.

26. The device of claim 25, further comprising an expansion balloon pre-loaded in at least the main body portion and the first limb portion such that the main body portion and the first limb portion are crimped around the expansion balloon in a predeployment state.

27. The device of claims 25-26, wherein the expansion element is configured to at least partially expand the second limb portion of the stent as the expansion element is inflated.

28. The device of any one of claims 26-27, wherein the wherein the expansion balloon is a stepped balloon, wherein a distal portion of the stepped balloon is positioned within the main body portion of has a larger expanded diameter than a more proximal portion of the expansion balloon which is positioned within the first limb portion.

29. The device of any one of claims 26-27, wherein the expansion balloon extends the entire length of the body.

30. The device of any one of claims 25-29, wherein the expansion balloon extends from an end of the body portion to an end of a limb portion.

31. The device of any one of claims 25-30, wherein the expansion balloon extends from the proximal end of the body portion to the distal end of the first limb portion.

32. The device of any one of claims 25-30, wherein the expansion element extends the entire length of the second limb.

33. The device of any one of claims 25-30, wherein the expansion element extends from an end of the first limb to an end of the second limb.

34. The device of any one of claims 25-30, wherein the expansion element extends from a distal end of the first limb to a distal end of the second limb.

35. The device of any one of claims 25-34, wherein the expansion element comprises and inner shaft and an outer shaft.
